(19) United States
(12) Patent Application Publication
(10) Pub. No.: US 2023/0068020 A1
(43) Pub. Date: Mar. 2, 2023

(54) MACROLIDE COMPOUND AND ITS USE OF TREATMENT CHRONIC RESPIRATORY DISEASE

(71) Applicant: BEIJING CONTINENT PHARMACEUTICALS CO., LTD., Beijing (CN)

(72) Inventors: Ying LUO, Shanghai (CN); Yanping YE, Shanghai (CN); Jianguo YU, Shanghai (CN)

(21) Appl. No.: 17/757,555

(22) PCT Filed: Jan. 8, 2020
(86) PCT No.: PCT/CN2020/070981

§ 371 Date: Jun. 16, 2022

(51) Int. Cl. C07D 407/12; C07D 413/12; C07D 493/08

(52) U.S. Cl. CPC C07D 407/12; C07D 413/12; C07D 493/08

(57) ABSTRACT

Macrolide compound and its use of treatment chronic respiratory disease are provided. Specifically, the macrolide compound is of formula (I) or pharmaceutically acceptable salts, stereoisomers and application thereof are effective in treating chronic respiratory disease.

### FIELD OF INVENTION

The present invention belongs to the field of medical technology and pharmaceuticals, and specifically, relates to macrolide compound and its use of treatment chronic respiratory disease.

### BACKGROUND

Chronic respiratory diseases are chronic diseases of airways and other structures of the lung. They are characterized by a high inflammatory cell recruitment and/or destructive cycle of infection. The most common chronic airway diseases are asthma, chronic obstructive pulmonary disease (COPD), and occupational lung diseases and pulmonary hypertension.

Chronic obstructive pulmonary disease (COPD) is a leading cause of death and disability worldwide. The Global Burden of Disease study has concluded that COPD will become the third leading cause of death worldwide by 2020, and will increase its ranking of disability-adjusted life years lost from 12th to 5th. COPD is a syndrome that encompasses both emphysema and chronic bronchitis that is most often caused by cigarette smoke. The disease is characterized by mucus accumulation, a robust immune response, and in later stages of the disease, chronic neutrophilia.

Macrolide anti-biotics have been used effectively and safely for the treatment of chronic respiratory infections for more than 60 years. Macrolide antibiotics typically used in clinic are characterized by the presence of a macrocyclic lactone ring containing 14 or 15 atoms to which one or two sugars are attached via glycosidic bonds.

It is an urgent need in the art to develop a new class of macrolide drugs with lower toxic, lower side effects and excellent anti-inflammatory effects, but without antibacterial activity.

### SUMMARY OF INVENTION

The object of the present invention is to provide a class of macrolide drug with lower toxic and side effects and excellent anti-inflammatory effects, which is useful for treatment in chronic respiratory disease and inflammatory disease.

In the first aspect of the present invention, it provides a compound of formula I, or a pharmaceutically acceptable salt, a stereoisomer thereof;

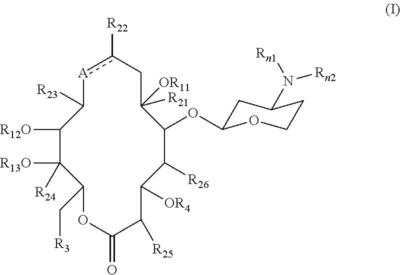

Wherein,
Rₙ₁ is selected from the group consisting of H, C₁₋₆ alkyl (preferably methyl);
Rₙ₂ is a substituted or unsubstituted group selected from the group consisting of H, C₁₋₁₀ alkyl (preferably, C₁₋₆ alkyl; more preferably, C₁₋₄ alkyl), —C₁₋₄ alkylene-C₆₋₁₀ aryl, —C₁₋₄ alkylene-(5- to 10-membered heteroaryl), C₁₋₆ alkanoyl (C₁₋₆ alkyl-C(O)—), —C₁₋₆ alkanoyl-C₆₋₁₀ aryl, —C₁₋₆alkanoyl-(5- to 10-membered heteroaryl), C₁₋₆ alkoxycarbonyl (C₁₋₆ alkyl-OC(O)—), —C₁₋₆ alkoxycarbonyl-C₆₋₁₀ aryl, —C₁₋₆ alkoxycarbonyl-(5- to 10-membered heteroaryl), C₂₋₁₀ alkenyl, and C₂₋₁₀ alkynyl;
R₁₂ and R₁₃ are independently selected from the group consisting of H, substituted or unsubstituted C₁₋₆ alkyl, substituted or unsubstituted C₃₋₆ cycloalkyl, R₅—C(O)—, and R₅—OC(O)—;
R₂₁, R₂₂, R₂₃, R₂₄, R₂₅ and R₂₆ are independently selected from the group consisting of H, and Substituted or unsubstituted C₁₋₆ alkyl (preferably methyl);
R₄ is selected from the group consisting of: H, substituted or unsubstituted C₁₋₆ alkyl, substituted or unsubstituted C₃₋₆ cycloalkyl, R₅—C(O)—, R₅—OC(O)—, and

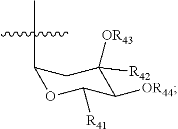

wherein,
R₄₁ and R₄₂ are independently selected from the group consisting of: H, substituted or unsubstituted C₁₋₆ alkyl;
R₄₃ is selected from the group consisting of: H, substituted or unsubstituted C₁₋₆ alkyl, Substituted or unsubstituted C₁₋₆ alkanoyl;
R₄₄ is selected from the group consisting of: H, substituted or unsubstituted C₁₋₆ alkyl, Substituted or unsubstituted C₁₋₆ alkanoyl; [symbol]
   is a double bond or single bond;
R₁₁ is selected from the group consisting of H, substituted or unsubstituted C₁₋₆ alkyl, substituted or unsubstituted C₃₋₆ cycloalkyl, R₅—C(O)—, and R₅—OC(O)—; or R₁₁ is null, and when R₁₁ is null, a single bond is formed between O to which R₁₁ attached and A;
A is selected from the group consisting of —C(O)—, —N(R₆)—(C(R′)₂)—, —CR′(R₇)—, —C(═N(OR₈))—,

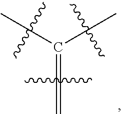 [SMILES: *C(*)=*]

### —CR′═, —CR′—;

R₆ is selected from the group consisting of H, Substituted or unsubstituted C₁₋₆ alkyl;
R₇ is selected from the group consisting of: H, —OH, Substituted or unsubstituted C₁₋₆ alkyl, Substituted or unsubstituted C₁₋₆ alkoxy, Substituted or unsubstituted C₁₋₆ alkyl-C(O)O—, Substituted or unsubstituted —N(R′)₂;
R₈ is selected from the group consisting of H, —C₁₋₆ alkyl, —C₁₋₄ alkylene-C₂₋₆ alkenyl, —C₁₋₄ alkylene-C₂₋₆ alkynyl, —C₁₋₄ alkylene-O—C₁₋₆ alkyl, —C₁₋₄ alkylene-S—C₁₋₆ alkyl, —C₁₋₄ alkylene-O—C₁₋₄ alkylene-O—C₁₋₆ alkyl; wherein R₈ can further be optionally substituted with a substituent selected from the group consisting of —OH, —CN, substituted or unsubstituted C₁₋₆ alkyl, substituted or unsubstituted C₆₋₁₀ aryl, substituted or unsubstituted 5- to 10-membered heteroaryl, substituted or unsubstituted —N(R′)₂, substituted or unsubstituted C₅₋₇ heterocycloalkyl, substituted or unsubstituted C₃₋₈ cycloalkyl;
R₅ is selected from the group consisting of: H, substituted or unsubstituted C₁₋₆ alkyl, substituted or unsubstituted —C₁₋₆ alkylene-C₆₋₁₀ aryl, substituted or unsubstituted 5- to 10-membered heteroaryl;
R′ is selected from the group consisting of: H, substituted or unsubstituted C₁₋₆ alkyl;
unless otherwise specified, the term “substituted” refers to one or more (preferably 1, 2, 3, 4 or 5) hydrogen in the group is substituted with a substituent selected from the group consisting of D, halogen, —OH, C₁₋₆ alkyl, C₁₋₆ haloalkyl.

In another preferred embodiment, when A is —C(O)—, Rₐ₁ and Rₐ₂ are different.

In another preferred embodiment, when A is —C(O)—, Rₙ₂ is not methyl.

In another preferred embodiment, when A is —C(O)—, Rₙ₂ is a substituted or unsubstituted group selected from the group consisting of C₁₋₆ alkanoyl (C₁₋₆ alkyl-C(O)—), —C₁₋₆ alkanoyl-C₆₋₁₀ aryl, —C₁₋₆alkanoyl-(5 - to 10-membered heteroaryl).

In another preferred embodiment, when A is selected from the group consisting of —N(R₆)—(C(R′)₂)—, —CR′(R₇)—, —C(═N(OR₈))—,

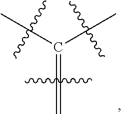 [SMILES: *C(*)=*]

—CR′═, —CR′—; Rₙ₂ is a substituted or unsubstituted group selected from the group consisting of H, C₁₋₁₀ alkyl, —C₁₋₄ alkylene-C₆₋₁₀ aryl, —C₁₋₄ alkylene-(5- to 10-membered heteroaryl), C₁₋₆ alkanoyl (C₁₋₆ alkyl-C(O)—), —C₁₋₆ alkanoyl-C₆₋₁₀ aryl, —C₁₋₆alkanoyl-(5- to 10-membered heteroaryl) C₁₋₆ alkoxycarbonyl (C₁₋₆ alkyl-OC(O)—), —C₁₋₆ alkoxycarbonyl-C₆₋₁₀ aryl, —C₁₋₆alkoxycarbonyl-(5- to 10-membered heteroaryl), C₂₋₁₀ alkenyl, and C₂₋₁₀ alkynyl.

In another preferred embodiment, the compound of Formula I is not LY101-2.

In another preferred embodiment, when

[symbol]

is a double bond, A is selected from the group consisting of

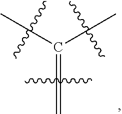 [SMILES: *C(*)=*]

### —CR′═.

In another preferred embodiment, when

[symbol]

is a single bond, A is selected from the group consisting of —C(O)—, —N(R₆)—(C(R′)₂)—, —CR′(R₇)—, —C(═N(OR₈))—.

In another preferred embodiment, when R₁₁ is null, A is

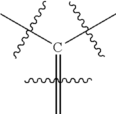 [SMILES: *C(*)=*]

### or, —CR′—.

In another preferred embodiment, when R₁₁ is not null, A is selected from the group consisting of —C(O)—, —N(R₆)—(C(R′)₂)—, —CR′(R₇)—, —C(═N(OR₈))—.

In another preferred embodiment, Rₙ₁ is H or methyl.

In another preferred embodiment, Rₙ₂ is selected from the group consisting of: H, C₁₋₁₀ alkyl (preferably, C₁₋₆ alkyl), and C₁₋₆ alkanoyl.

In another preferred embodiment, Rₙ₂ is C₁₋₆ alkanoyl; preferably, C₁₋₄ alkanoyl.

In another preferred embodiment, the compound of formula I has a structure of formula I-i;

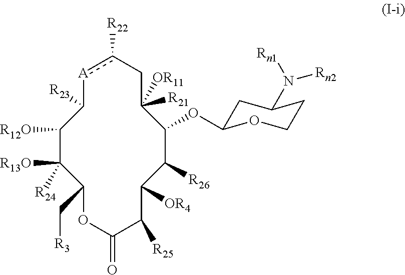

Wherein,

[symbol]

, Rₙ₁, Rₙ₂, R₁₁, R₁₂, R₁₃, R₂₁, R₂₂, R₂₃, R₂₄, R₂₅, R₂₆, R₃, R₄, and A are defined as above.

In another preferred embodiment, the compound of formula I has a structure of formula Ia, Ib, Ic, Id or Ie,

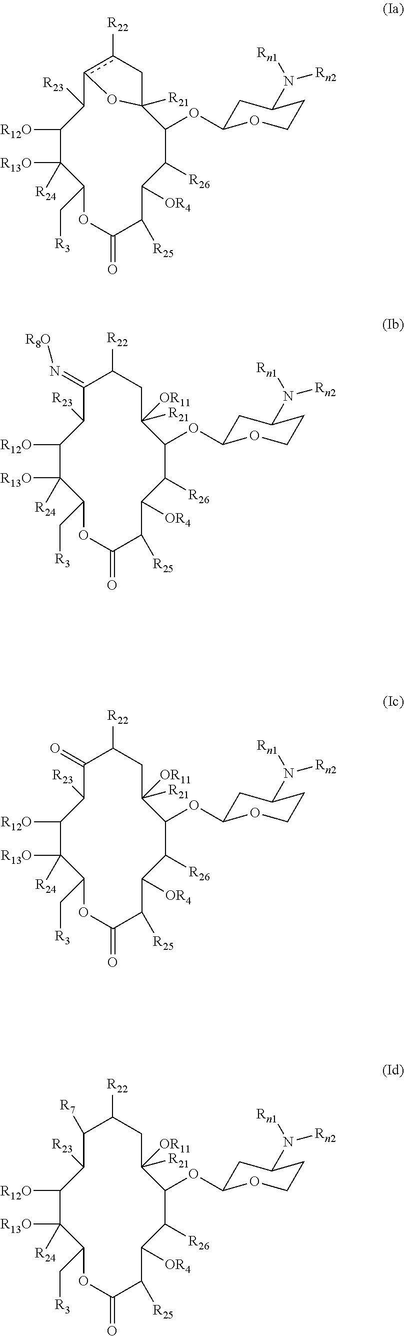

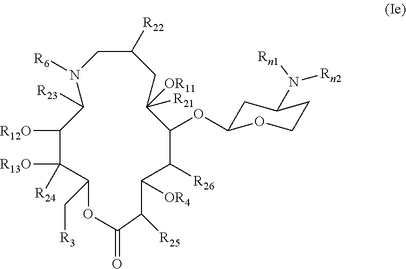

wherein, Rₙ₁, Rₙ₂, R₁₁, R₁₂, R₁₃, R₂₁, R₂₂, R₂₃, R₂₄, R₂₅, R₂₆, R₃, R₄, R₆, R₇ and R₈ are defined as above.

In another preferred embodiment, the compound of formula I has a structure of Ia-i, Ib-i, Ic-i, Id-i or Ie-I,

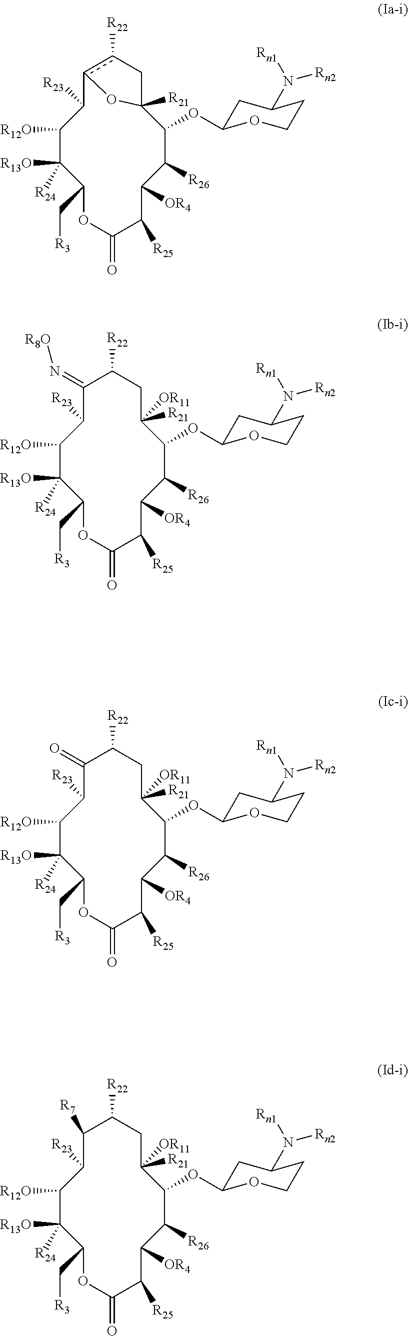

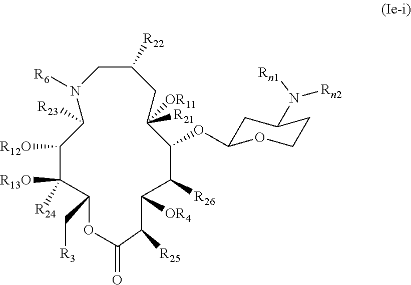

wherein, [symbol]
   , Rₙ₁, Rₙ₂, R₁₁, R₁₂, R₁₃, R₂₁, R₂₂, R₂₃, R₂₄, R₂₅, R₂₆, R₃, R₄, R₆, R₇ and R₈ are defined as above.

In another preferred embodiment, when the compound of formula I has a structure of Ic or Ic-i, Rₙ₁ and Rₙ₂ are different.

In another preferred embodiment, when the compound of formula I has a structure of Ic or Ic-i, Rₙ₂ is not methyl.

In another preferred embodiment, when the compound of formula I has a structure of Ic or Ic-i, Rₙ₂ is a substituted or unsubstituted group selected from the group consisting of C₁₋₆ alkanoyl, —C₁₋₆ alkanoyl-C₆₋₁₀ aryl, —C₁₋₆alkanoyl-(5- to 10-membered heteroaryl); preferably, Rₙ₂ is a substituted or unsubstituted C₁₋₆ alkanoyl.

In another preferred embodiment, when the compound of formula I has a structure of Ia, Ib, Id, Ie, Ia-i, Ib-i, Id-I, or Ie-I;
Rₙ₂ is a substituted or unsubstituted group selected from the group consisting of H, C₁₋₁₀ alkyl, —C₁₋₄ alkylene-C₆₋₁₀ aryl, —C₁₋₄ alkylene-(5- to 10-membered heteroaryl), C₁₋₆ alkanoyl (C₁₋₆ alkyl-C(O)—), —C₁₋₆ alkanoyl-C₆₋₁₀ aryl, —C₁₋₆alkanoyl-(5- to 10-membered heteroaryl) C₁₋₆ alkoxycarbonyl (C₁₋₆ alkyl-OC(O)—), —C₁₋₆ alkoxycarbonyl-C₆₋₁₀ aryl, —C₁₋₆alkoxycarbonyl-(5- to 10-membered heteroaryl), C₂₋₁₀ alkenyl, and C₂₋₁₀ alkynyl.

In another preferred embodiment, when the compound of formula I has a structure of Ia, Ib, Id, Ie, Ia-i, Ib-i, Id-I, or Ie-I; Rₙ₂ is a substituted or unsubstituted group selected from the group consisting of H, C₁₋₁₀ alkyl, C₁₋₆ alkanoyl, and C₁₋₆ alkoxycarbonyl.

In another preferred embodiment, [symbol] , Rₙ₁, Rₙ₂, R₁₁, R₁₂, R₁₃, R₂₁, R₂₂, R₂₃, R₂₄, R₂₅, R₂₆, R₃, R₄,

R₆, R₇ and R₈ are the corresponding groups in the compounds as prepared in the Examples.

In another preferred embodiment, the compound of formula (I) is any of compounds listed in Table A.

In the second aspect of the present invention, it provides a pharmaceutical composition comprising a compound of the first aspect of the present invention, or a pharmaceutically acceptable salt, a stereoisomer thereof; and a pharmaceutically acceptable carrier.

In the third aspect of the present invention, it provides a use of a compound of the first aspect of the present invention, or a pharmaceutically acceptable salt, a stereoisomer thereof for manufacture of a medicament for treating or preventing inflammatory disease.

In another preferred embodiment, the inflammatory disease is chronic inflammatory disease.

In another preferred embodiment, the inflammatory disease is a chronic respiratory inflammatory disease.

In another preferred embodiment, the inflammatory disease is selected from the group consisting of: chronic obstructive pulmonary disease (COPD), asthma, diffuse panbronchiolitis, cystic pulmonary fibrosis, bronchiectasis, or a combination thereof.

In the fourth aspect of the present invention, it provides a method for treating or preventing inflammatory disease, which comprise a step of:
administering a compound according to any one of the first aspect of the present invention or a pharmaceutical composition of the second aspect of the present invention to a subject in need.

In another preferred embodiment, the subjects comprises human and non-human mammal.

In another preferred embodiment, the inflammatory disease is chronic inflammatory disease.

In another preferred embodiment, the inflammatory disease is a chronic respiratory inflammatory disease.

In another preferred embodiment, the inflammatory disease is selected from the group consisting of: chronic obstructive pulmonary disease (COPD), asthma, diffuse panbronchiolitis, cystic pulmonary fibrosis, bronchiectasis, or a combination thereof.

In the fifth aspect of the present invention, it provides a method for promoting monocyte to macrophage in vitro, which comprises a step of: culturing cell in the present of a compound of the first aspect of the present invention.

In another preferred embodiment, the cell comprises THP-1 cell.

In the sixth aspect of the present invention, it provides a method for inhibiting the IL-8 expression in vitro, which comprises a step of: culturing cell in the present of a compound of the first aspect of the present invention.

In another preferred embodiment, the cell comprises BEAS-2B cell.

It should be understood that each of the above technical features of the invention and each technical feature specifically described below (such as in Examples) can be combined with each other within the scope of the present invention so as to constitute new or preferred technical solutions.

### BRIEF DESCRIPTION OF THE DRAWINGS

FIG. 1 shows the effect of Compound A on differentiation of THP-1 cell to macrophages.

FIG. 2 shows the effect of Compound A on LPS induced release of IL-8 by BEAS-2B.

FIG. 3 shows the effect of Compound A on elastase induced emphysema mice model—Representative HE staining on sagittal sections of the left lung (100× original magnification).

FIG. 4 shows the effect of Compound A on elastase induced emphysema mice model—the mean chord length of alveoli measured from histopathological images in FIG. 3.

FIG. 5 shows the effect of Compound A on smoke induced COPD mice model—Representative HE staining on sagittal sections of the left lung (100× original magnification)

FIG. 6 shows the effect of Compound A on smoke induced COPD mice model the mean chord length of alveoli measured from histopathological images in FIG. 5.

FIG. 7 shows the effect of Compound A on smoke induced COPD mice model—Number of total inflammatory cells in BALF, number of macrophages and number of neutrophils.

FIG. 8 shows the effect of Compound A on smoke induced COPD mice model—total lung capability and airway resistance.

### DETAILED DESCRIPTION OF INVENTION

After extensive and intensive research, the inventors have unexpectedly developed a class of novel compound of formula I (have a group of

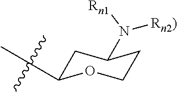

that show lower toxic, lower side effects and excellent anti-inflammatory effects, but without antibacterial activity. The compound of invention is useful for treatment of chronic diseases (such as chronic obstructive pulmonary disease) and avoids unnecessary bacterial resistance. The present invention is completed on this basic.

Definition

As used herein, the term “alkyl”, by itself or as part of another substituent, means, unless otherwise stated, a straight or branched chain hydrocarbon radical, having the number of carbon atoms designated (i.e. C₁₋₁₀ means one to ten carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. Preferably, the alkyl group has 1 to 6 carbon atoms, i.e. C₁₋₆ alkyl; more preferably, the alkyl group has 1 to 4 carbon atoms, i.e. C₁₋₄ alkyl.

As used herein, the term “alkenyl” refers to an unsaturated alkyl group having one or more double bonds. Similarly, the term “alkynyl” refers to an unsaturated alkyl group having one or more triple bonds. Examples of such unsaturated alkyl groups include vinyl, 2-propenyl, crotyl, 2-isopentenyl, 2-(butadienyl), isobutenyl, 2,4-pentadienyl, 3-(1,4-pentadienyl), ethynyl, 1- and 3-propynyl, 3-butynyl, and the higher homologs and isomers.

As used herein, the term “cycloalkyl” refers to hydrocarbon rings having the indicated number of ring atoms (e.g., C₃₋₆cycloalkyl) and being fully saturated or having no more than one double bond between ring vertices. “Cycloalkyl” is also meant to refer to bicyclic and polycyclic hydrocarbon rings such as, for example, bicyclo[2.2.1]heptane, bicyclo[2.2.2]octane, etc.

As used herein, the term “heterocycloalkyl” refers to a cycloalkyl group that contain from one to five heteroatoms selected from N, O, and S, wherein the nitrogen and sulfur atoms are optionally oxidized, and the nitrogen atom (s) are optionally quaternized. The heterocycloalkyl may be a monocyclic, a bicyclic or a polycylic ring system. Non limiting examples of heterocycloalkyl groups include pyrrolidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrhydrothiophene, quinuclidine, and the like. A heterocycloalkyl group can be attached to the remainder of the molecule through a ring carbon or a heteroatom.

As used herein, the term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms, with those groups having 10 or fewer (more preferably, 1 to 6, or 1 to 4) carbon atoms being preferred in the present invention. A “lower alkyl” or “lower alkylene” is a shorter chain alkyl or alkylene group, generally having four or fewer carbon atoms. Similarly, “alkenylene” and “alkynylene” refer to the unsaturated forms of “alkylene” having double or triple bonds, respectively.

As used herein, the term “heteroalkylene” by itself or as part of another substituent means a divalent radical, saturated or unsaturated or polyunsaturated, derived from heteroalkyl, as exemplified by —CH₂-CH₂-S—CH₂CH₂— and

—CH₂—S—CH₂—CH₂—NH—CH₂—, —O—CH₂—CH═CH—, —CH₂—CH═C(H)CH₂—O—CH₂— and —S—CH₂—C≡C—. For heteroalkylene groups, heteroatoms can also occupy either or both of the chain termini (e.g., alkyleneoxy, alkylenedioxy, alkyleneamino, alkylenediamino, and the like).

As used herein, the terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, and the like.

As used herein, the term “aryl” means, unless otherwise stated, a polyunsaturated, typically aromatic, hydrocarbon group which can be a single ring or multiple rings (up to three rings) which are fused together or linked covalently.

As used herein, the term “heteroaryl” refers to aryl groups (or rings) that contain from one to five heteroatoms selected from N, O, and S, wherein the nitrogen and sulfur atoms are optionally oxidized, and the nitrogen atom (s) are optionally quaternized. A heteroaryl group can be attached to the remainder of the molecule through a heteroatom. Non-limiting examples of aryl groups include phenyl, naphthyl and biphenyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzooxazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, pyrrolopyridyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. Substituents for each of the above noted aryl and heteroaryl ring systems are selected from the group of acceptable substituents described below.

As used herein, Compound A means a compound selected from LY101-25, LY101-22, LY101-45, LY101-39, LY101-33, LY101-27 and LY101-48.

As used herein, “Cmpd” is the short for compound, similarly, “Cmpd A” is the short for Compound

A.

Unless otherwise specified, the abbreviations used herein represent conventional meanings known to those skilled in the art.

Pharmaceutical Composition

As used herein, term “the active material of the invention” or “the active compound of the invention” refers to the compound of formula (I) of the invention, or a pharmaceutically acceptable salt, solvate, stereoisomer, or prodrug thereof.

As used herein, “pharmaceutically acceptable salt (s)” includes pharmaceutically acceptable acid addition salt (s) and base addition salt (s).

As used herein, term “Pharmaceutically acceptable acid addition salts” refer to salts that are able to retain the biological effectiveness of the free base without other side effects and are formed with inorganic or organic acids. Inorganic acid salts include, but not limited to, hydrochloride, hydrobromide, sulfate, phosphate and the like; organic acid salts include, but not limited to, formate, acetate, propionate, glycolate, gluconate, lactate, oxalate, maleate, succinate, fumarate, tartrate, citrate, glutamate, aspartate, benzoate, methanesulfonate, p-toluenesulfonate, salicylate and the like. These salts can be prepared by the methods known in the art.

As used herein, term “Pharmaceutically acceptable base addition salts” include, but not limited to the salts of inorganic bases such as sodium, potassium, calcium and magnesium salts, and include but not limited to the salts of organic bases, such as ammonium salt, triethylamine salt, lysine salt, arginine salt and the like. These salts can be prepared by the methods known in the art.

As used herein, the compounds of formula (I) may exist in one or more crystalline forms. The active compounds of the present invention include various polymorphs and mixtures thereof.

The “solvate” mentioned in the present invention refers to a complex formed with the compound of the present invention and a solvent. The solvate can be formed either through a reaction in a solvent or precipitated or crystallized from the solvent. For example, a complex formed with water is referred to as “hydrate”. The solvates of the compounds of formula (I) are within the scope of the present invention.

The compounds of formula (I) of the invention may contain one or more chiral centers, and may exist in different optically active forms. When the compound contains one chiral center, the compound includes enantiomers. The present invention includes both of two isomers and a mixture thereof, such as racemic mixtures. Enantiomers can be resolved using methods known in the art, such as crystallization and chiral chromatography and the like. When the compound of formula (I) contain more than one chiral centers, the compounds may include diastereomers. The present invention includes specific isomers resolved into optically pure isomers as well as the mixtures of diastereomeric isomers. Diastereomeric isomers can be resolved using methods known in the art, such as crystallization and preparative chromatography.

The present invention includes prodrugs of the above-mentioned compounds. Prodrugs include known amino protecting groups and carboxyl protecting groups which are hydrolyzed under physiologic conditions or released by enzyme reaction to obtain the parent compounds. Specific preparation methods of prodrugs can refer to (Saulnier, M G; Frennesson, D B; Deshpande, M S; Hansel, S B and Vysa, D M Bioorg. Med. Chem Lett. 1994, 4, 1985-1990; and Greenwald, R B; Choe, Y H; Conover, C D; Shum, K.; Wu, D.; Royzen, M. J. Med. Chem. 2000, 43, 475).

As used herein, term “therapeutically effective amount” refers to an amount that yields a function or activity to humans and/or animals and may be tolerated by humans and/or animals.

The pharmaceutical composition provided by the present invention preferably contains the active ingredient in a weight ratio of 1 to 99%. Preferably, the compound of the general formula I accounts for 65wt % to 99wt % of the total weight as the active ingredient, and the rest are pharmaceutically acceptable carriers, diluents, solutions or salt solutions.

The compounds and pharmaceutical compositions provided by the present invention may be in various forms, such as tablets, capsules, powders, syrups, solutions, suspensions, aerosols, etc., and may be present in suitable solid or liquid carriers or diluents, and in disinfectors suitable for injection or instillation.

Various dosage forms of the pharmaceutical compositions of the present invention can be prepared according to the conventional preparation methods in the pharmaceutical field. The unit dosage of its formulation formula comprises 0.05-200 mg of the compound of formula I, preferably, the unit dosage of the formulation formula contains 0.1 mg-100 mg of the compound of formula I.

The compounds of the present invention may be administered alone or in combination with other pharmaceutically acceptable compounds (such as other ion channel inhibitors).

The compounds and pharmaceutical compositions of the present invention can be used clinically in mammals, including humans and animals, and can be administered via mouth, nose, skin, lung or gastrointestinal tract. Most preferred is oral. The most preferred daily dose is 0.01-200 mg/kg body weight in one dose, or 0.01-100 mg/kg body weight in divided doses. Regardless of the administering method, the individual's optimal dose should be based on the specific treatment. Usually, it starts with a small dose, which is gradually increased until the most suitable dose is found.

Preparation Method

The present invention provides preparation methods of compounds of formula (I). The compounds of the present invention can be easily prepared by a variety of synthetic operations, and these operations are familiar to those skilled in the art. An exemplary preparation of these compounds may include (but not limited to) the processes described below.

Generally, in the preparation process, each reaction is generally conducted in an inert solvent, under room temperature to reflux temperature (such as 0-150° C., preferably from 0-100° C.). The reaction time is usually 0.1 hours-60 hours, preferably 0.5 to 48 hours.

Preferably, compounds of formula (I) of the present invention can be prepared referring to any of the following schemes. The procedures of method can be extended or combined as desired in practice.

The Main Advantages of the Invention Include:
(a) The compounds of the present invention have lower toxicity.
(b) The compounds of the present invention have no antibacterial activity, and thus is unlikely to cause bacterial resistance and is suitable for treating chronic diseases.
(c) The compound of the present invention has excellent anti-inflammatory ability while having low toxicity and low antibacterial activity.
(d) In particular, the compounds of the present invention, especially those in Table A, and more especially Compound A, have a lower toxicity and lower antibacterial activity, excellent anti-inflammatory ability, and an excellent wide therapeutic window.

The present invention will be further described below with reference to specific embodiments. It should be understood that these examples are only used to illustrate the present invention and not to limit the scope of the present invention. The experimental methods without specific conditions in the following examples are generally based on conventional conditions or conditions recommended by the manufacturer. Unless stated otherwise, percentages and parts are percentages by weight and parts by weight.

### EXAMPLE 1 and 2

### Synthesis of Ly101-25 and ly-101-22

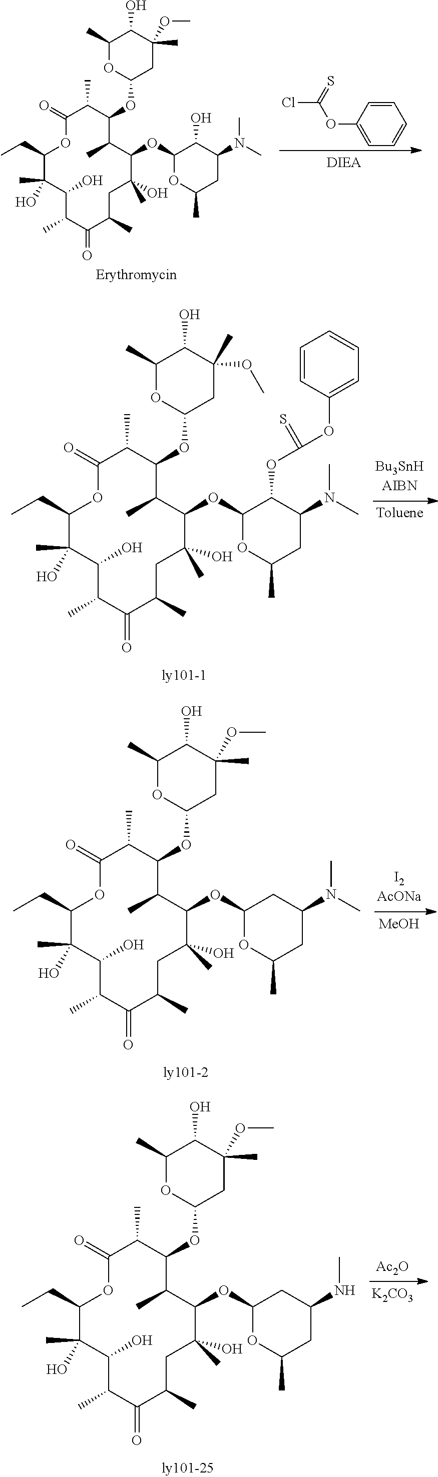

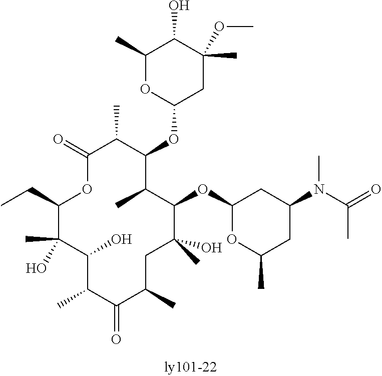 [SMILES: CC[C@H]1OC(=O)[C@H](C)[C@@H](O[C@H]2C[C@@](C)(OC)[C@@H](O)[C@H](C)O2)[C@H](C)[C@@H](O[C@H]2C[C@@H](N(C)C(C)=O)C[C@@H](C)O2)[C@](C)(O)C[C@@H](C)C(=O)[C@H](C)[C@@H](O)[C@]1(C)O]

Step 1: synthesis of

O-((2S,3R,4S,6R)-4-(dimethylamino)-2-(((3R,4S,5S,6R,7R,9R,11R,12R,13S,14R)-14-ethyl-7,12,13-trihydroxy-4-(((2R,4R,5S,6S)-5-hydroxy-4-methoxy-4,6-dimethyltetrahydro-2H-pyran-2-yl)oxy)-3,5,7,9,11,13-hexamethyl-2,10-dioxooxacyclotetradecan-6-yl)oxy)-6-methyltetrahydro-2H-pyran-3-yl) O-phenyl carbonothioate(ly101-1)

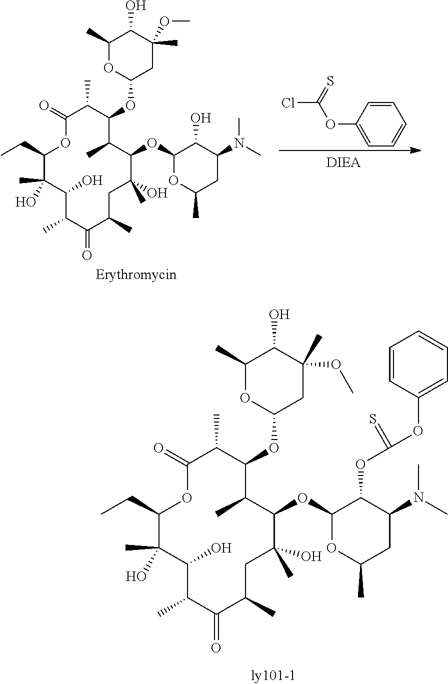 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2O)[C@](C)(O)C[C@@H](C)C(=O)[C@H](C)[C@@H](O)[C@]1(C)O ; S=C(Cl)Oc1ccccc1 ; CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2OC(=S)Oc2ccccc2)[C@](C)(O)C[C@@H](C)C(=O)[C@H](C)[C@@H](O)[C@]1(C)O]

To a solution of Erythromycin (5 g, 6.8 mmol) in DCM (250 ml) was slowly added DIEA (1.05 g, 8.1 mmol) and phenyl chlorothionocarbonate (1.25 g, 7.3 mmol) at 5° C. The mixture was maintained at RT for 3 h. TLC showed that no raw material left. The mixture was concentrated, and the resulting residue was purified by silica-gel column chromatography eluting with ethyl acetate in petroleum (0% to 40%) to give ly101-1 (3.7 g, yield: 62.5%) as a white solid. MS (ESI) m/z: 870 [M+H]⁺. 1H NMR (400 MHz, CDCl₃) δ 7.44-7.31 (m, 2H), 7.27 (d, J=7.3 Hz, 1H), 7.12 (dd, J=23.9, 7.6 Hz, 2H), 5.32 (dd, J=10.5, 7.2 Hz, 1H), 5.04 (d, J=8.9 Hz, 1H), 4.88 (d, J=4.4 Hz, 1H), 4.62 (dd, J=17.0, 7.1 Hz, 1H), 3.93 (d, J=3.6 Hz, 3H), 3.79 (s, 1H), 3.51 (t, J=7.5 Hz, 2H), 3.26-3.16 (m, 3H), 3.13 (s, 1H), 3.03 (dd, J=13.4, 7.6 Hz, 2H), 2.86 (dd, J=14.2, 7.5 Hz, 2H), 2.63 (s, 1H), 2.42-2.12 (m, 9H), 2.02 (s, 1H), 1.98-1.82 (m, 3H), 1.78 (s, 1H), 1.66 (s, 1H), 1.64-1.55 (m, 2H), 1.55-1.32 (m, 6H), 1.24 (dd, J=12.9, 6.0 Hz, 11H), 1.09 (dd, J=16.1, 9.3 Hz, 7H), 1.03 (d, J=7.5 Hz, 3H), 0.83 (t, J=7.4 Hz, 3H).

Step 2: synthesis of

(3R,4S,5S,6R,9R,11R,12R,13S,14R)-6-(((2S,4S,6R)-4-(dimethylamino)-6-methyltetrahydro-2H-pyran-2-yl)oxy)-14-ethyl-7,12,13-trihydroxy-4-(((2R,4R,5S,6S)-5-hydroxy-4-methoxy-4,6-dimethyltetrahydro-2H-pyran-2-yl)oxy)-3,5,7,9,11,13-hexamethyloxacyclotetradecane-2,10-dione (ly101-2)

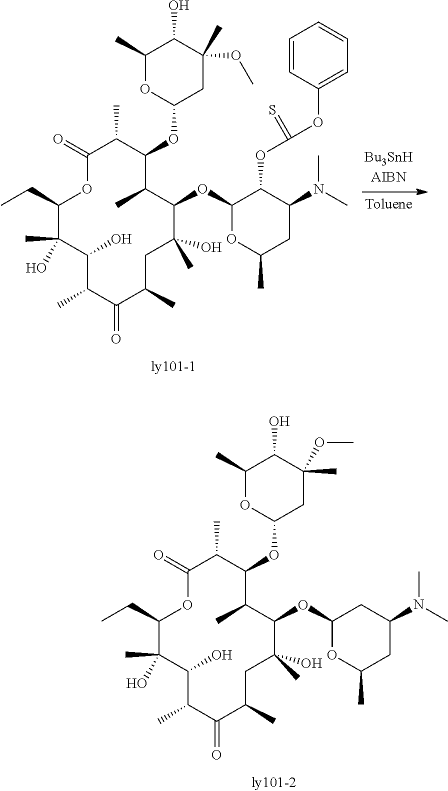 [SMILES: CC[C@H]1OC(=O)[C@H](C)[C@@H](O[C@H]2C[C@@](C)(OC)[C@@H](O)[C@H](C)O2)[C@H](C)[C@@H](O[C@@H]2O[C@H](C)C[C@H](N(C)C)[C@H]2OC(=S)Oc2ccccc2)[C@](C)(O)C[C@@H](C)C(=O)[C@H](C)[C@@H](O)[C@]1(C)O ; CC[C@H]1OC(=O)[C@H](C)[C@@H](O[C@H]2C[C@@](C)(OC)[C@@H](O)[C@H](C)O2)[C@H](C)[C@@H](O[C@H]2C[C@@H](N(C)C)C[C@@H](C)O2)[C@](C)(O)C[C@@H](C)C(=O)[C@H](C)[C@@H](O)[C@]1(C)O]

To a solution of ly101-1 (7.5 g, 8.6 mmol) in toluene (250 ml) was added AIBN (1.54 g, 9.4 mmol) and Bu3SnH (7.5 g, 26 mmol). The mixture was maintained at 90° C. for 3 h. TLC analysis showed that raw material was completely consumed. The mixture was concentrated, and the resulting residue was purified by silica-gel column chromatography, eluting with methanol in ethyl acetate (0% to 10%), to give ly101-2 (4 g, yield: 64.5%) as a white solid. MS (ESI) m/z: 729.1 [M+H]⁺. 1H NMR (400 MHz, CDCl3) δ 5.05 (d, J=10.8 Hz, 1H), 4.87 (d, J=5.0 Hz, 1H), 4.50 (d, J=9.6 Hz, 1H), 4.12 (dd, J=14.2, 7.1 Hz, 1H), 3.94 (dd, J=16.4, 9.6 Hz, 3H), 3.80 (s, 1H), 3.54 (d, J=7.6 Hz, 1H), 3.40 (s, 1H), 3.28 (d, J=11.4 Hz, 3H), 3.10 (s, 2H), 3.02 (t, J=9.6 Hz, 1H), 2.93-2.78 (m, 1H), 2.69 (s, 1H), 2.39 (dd, J=21.5, 13.6 Hz, 2H), 2.31-2.15 (m, 6H), 2.05 (s, 2H), 1.97-1.80 (m, 3H), 1.74-1.55 (m, 8H), 1.33 (dd, J=15.8, 9.3 Hz, 2H), 1.30-1.26 (m, 3H), 1.23 (d, J=9.1 Hz, 6H), 1.20-1.10 (m, 11H), 0.99-0.88 (m, 3H), 0.85 (t, J=7.3 Hz, 3H).

Step 3: synthesis of (3R,4S,5S,6R,9R,11R,12R,13S,14R)-14-ethyl-7,12,13-trihydroxy-4-(((2R,4R,5S,6S)-5-hydroxy-4-methoxy-4,6-dimethyltetrahydro-2H-pyran-2-yl)oxy)-3,5,7,9,11,13-hexamethyl-6-(((2S,4S,6R)-6-methyl-4-(methylamino)tetrahydro-2H-pyran-2-yl)oxy)oxacyclotetradecane-2,10-dione(ly101-25)

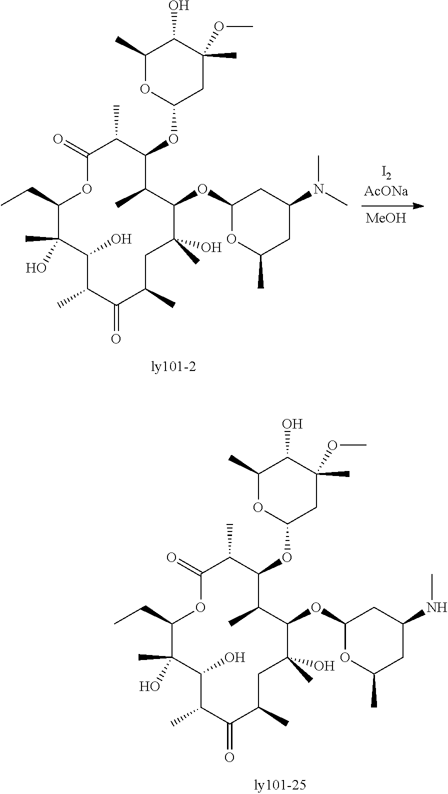

To a stirred solution of ly101-2 (5 g, 6.8 mmol) in methanol (150 ml) and water (30 ml) was added sodium acetate (2.9 g, 35.3 mmol) and solid iodine (2.3 g, 9 mmol). Then the solution was maintained between pH 8-9, followed by addition of 1N aqueous NaOH. The mixture was stirred at 50° C. for 3 h. TLC analysis showed that raw material was completely consumed. The mixture was concentrated, and the resulting residue was purified by silica-gel column chromatography eluting with methanol in ethyl acetate (0% to 10%) to give ly101-25 (1.8 g, yield: 37.2%) as a white solid. MS (ESI) m/z: 705.5 [M+H]⁺. H NMR (400 MHz, CDCl3) δ 5.05 (dd, J=11.0, 2.1 Hz, 1H), 4.88 (t, J=6.2 Hz, 1H), 4.52 (d, J=7.8 Hz, 1H), 4.08-3.86 (m, 2H), 3.80 (d, J=7.7 Hz, 1H), 3.62 (d, J=8.4 Hz, 1H), 3.54 (d, J=7.6 Hz, 1H), 3.50-3.38 (m, 2H), 3.34-3.23 (m, 3H), 3.06 (dd, J=24.7, 8.2 Hz, 3H), 2.90-2.76 (m, 2H), 2.67 (d, J=6.9 Hz, 1H), 2.54 (s, 2H), 2.35 (d, J=14.9 Hz, 2H), 2.24 (d, J=9.5 Hz, 1H), 2.06-1.75 (m, 5H), 1.60 (dd, J=23.9, 13.7 Hz, 3H), 1.52-1.37 (m, 5H), 1.32-1.20 (m, 10H), 1.20-1.06 (m, 11H), 0.97 (t, J=13.1 Hz, 3H), 0.85 (dd, J=14.3, 7.1 Hz, 3H). 13C NMR (101 MHz, DMSO) δ 218.45, 175.09, 146.06, 110.05, 101.88, 86.05, 83.85, 79.56, 77.78, 76.26, 74.95, 73.51, 73.11, 69.33, 67.64, 65.19, 54.99, 49.51, 44.82, 38.55, 37.94, 33.23, 26.96, 22.06, 21.65, 21.30, 18.85, 18.64, 17.87, 11.90, 11.08, 9.61.

Step 4: synthesis of

N-((2S,4S,6R)-2-(((3R,4S,5S,6R,7R,9R,11R,12R,13S,14R)-14-ethyl-7,12,13-trihydroxy-4-(((2R,4R,5S,6S)-5-hydroxy-4-methoxy-4,6-dimethyltetrahydro-2H-pyran-2-yl)oxy)-3,5,7,9,11,13-hexamethyl-2,10-dioxooxacyclotetradecan-6-yl)oxy)-6-methyltetrahydro-2H-pyran-4-yl)-N-methylacetamide (ly101-22)

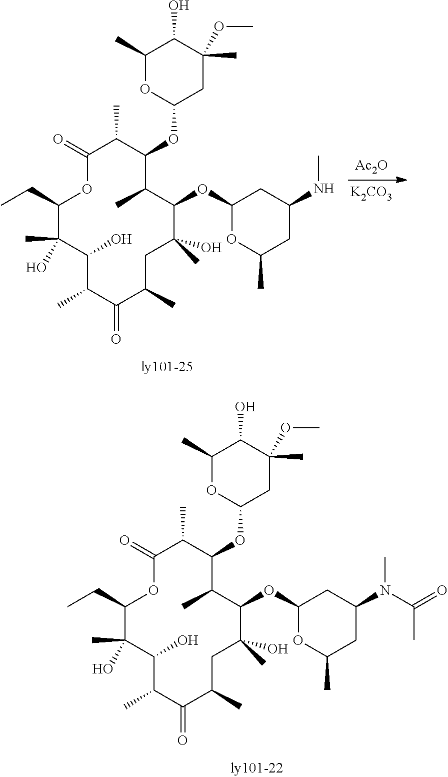

To a stirred solution of ly101-25 (3 g, 4.3 mmol) in 1,4-Dioxane (60 ml) and water (60 ml) was added acetic anhydride (673 mg, 6.6 mmol) and Potassium carbonate (1.5 g, 11 mmol). The solution was stirred at RT for 3 h. TLC analysis showed that raw material was completely consumed. The mixture was concentrated and the resulting residue was purified by silica-gel column chromatography eluting with methanol in ethyl acetate (0% to 10%) to give ly101-22 (1.6 g, yield: 50%) as a white solid. MS (ESI) m/z: 768.4 [M+Na]⁺. 1H NMR (400 MHz, CDCl3) δ 5.11-4.99 (m, 1H), 4.88 (d, J=4.9 Hz, 1H), 4.81-4.68 (m, 1H), 4.67-4.55 (m, 1H), 4.05-3.86 (m, 3H), 3.81 (d, J=14.6 Hz, 1H), 3.54 (t, J=9.7 Hz, 2H), 3.39-3.26 (m, 3H), 3.21 (s, 1H), 3.14-3.05 (m, 2H), 3.01 (dd, J=17.4, 7.5 Hz, 1H), 2.88-2.78 (m, 3H), 2.74-2.60 (m, 1H), 2.50-2.30 (m, 2H), 2.16-2.06 (m, 3H), 1.86 (dd, J=34.5, 12.3 Hz, 4H), 1.72 (s, 2H), 1.67-1.53 (m, 3H), 1.45 (d, J=14.6 Hz, 2H), 1.33-1.21 (m, 10H), 1.21-1.07 (m, 14H), 0.94 (t, J=6.4 Hz, 3H), 0.88-0.77 (m, 3H). 13C NMR (101 MHz, DMSO) δ 218.02, 174.55, 169.35, 99.33, 95.95, 84.10, 78.86, 77.35, 75.79, 74.58, 72.98, 72.70, 68.77, 67.22, 64.82, 48.80, 47.28, 44.33, 39.93, 38.89, 37.90, 35.40, 35.01, 33.97, 29.67, 26.48, 22.23, 21.44, 20.80, 18.42, 18.19, 17.31, 15.73, 11.45, 10.56, 9.10.

### EXAMPLE 3

### Synthesis of Ly101-3

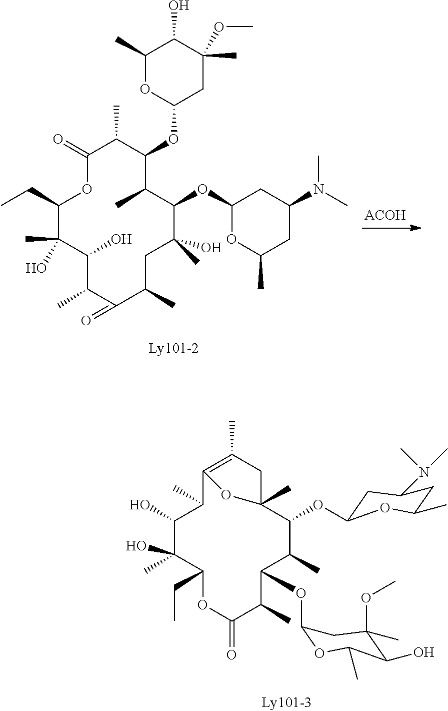

Ly101-2 (7 g, 10 mmol, 1.0 eq) was placed in a 250 mL three-neck flask and dissolved with AcOH (30 mL). The solution was stirred at RT for 2 h. TLC (DCM/MeOH/aq. Ammonia=10:100:1) that reaction was completed. The reaction mixture was added sat. NaHCO₃ (700 mL) dropwise until pH=8-9, extracted twice with DCM (200 mL), dried over anhydrous Na₂SO₄ and concentrated to obtain Ly101-3 (6 g, 85%). MS (ESI) m/z: 700.4 [M+H]⁺

### EXAMPLE 4

### Synthesis of Ly101-6

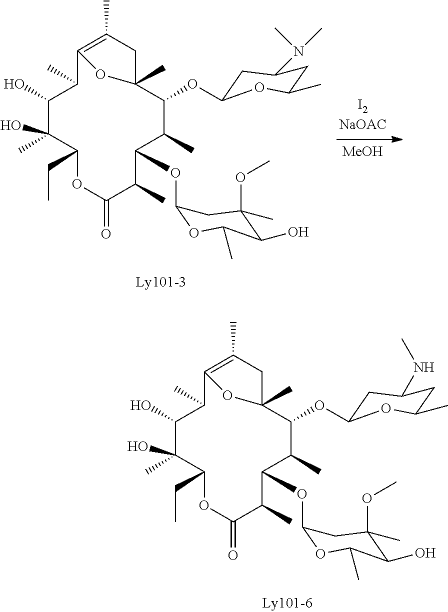

To a 50 mL single-neck flask was added Ly101-3 (170 mg, 0.25 mmol, 1.0 eq), iodine (89 mg, 0.35 mmol, 1.4 eq) and sodium acetate (117 mg, 1.4 mmol, 5.7 eq). It was dissolved with MeOH (6 ml) and water (1 mL). The solution was stirred at 50° C., adjusted to pH=8-9 using aq. NaOH (0.5 mol/L), and heated for 2 h. TLC (DCM/MeOH/aq. Ammonia=10:100:1) showed that reaction was completed. The reaction mixture was concentrated to dry, and purified by column to obtain Ly101-6 (50 mg, 29.1%). MS (ESI) m/z: 686.5 [M+H]⁺.

### EXAMPLE 5

### Synthesis of Ly101-24

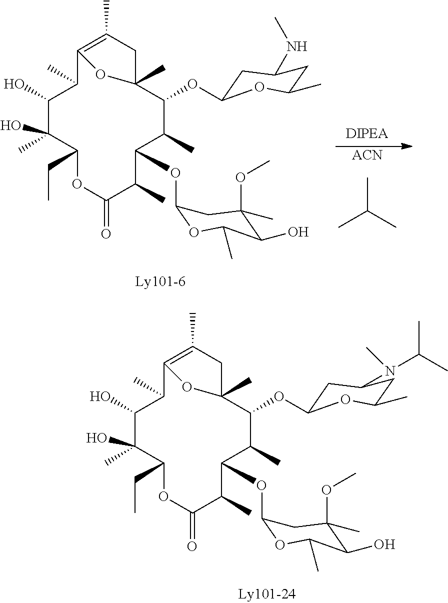

To a 50 mL single-neck flask was added Ly101-6 (0.5 g, 0.73 mmol, 1.0 eq), DIPEA (0.54 g, 4.1 mmol, 5.7 eq), and iso-propyl iodide (0.3 g, 1.7 mmol, 3.9 eq). It was dissolved with ACN (10 ml). The mixture was stirred at 77° C. for 15 h. TLC (DCM/MeOH/aq. Ammonia=10:100:1) showed that the reaction was completed. The reaction mixture was concentrated to dry and purified by column chromatography (DCM/MeOH) to obtain Ly101-24 (150 mg, 28.2%). MS (ESI) m/z: 728.8 [M+H]⁺.

### EXAMPLE 6

### Synthesis of Ly101-27

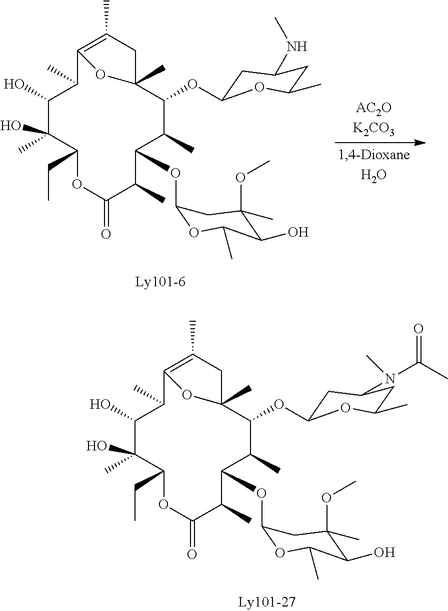

To a 50 ml single-neck flask was added Ly101-5 (0.5 g, 0.73 mmol, 1.0 eq), acetic anhydride (156.3 mg, 1.5 mmol, 2.1 eq), K₂CO₃ (0.5 g, 3.56 mmol, 5.0 eq), dioxane (5 mL), and water (5 mL). The mixture was stirred in ice bath for 30 min, then stirred for another 3 h after removing the ice bath. TLC (EA/MeOH/aq. Ammonia=3:1:0.15) showed the reaction was completed. The reaction mixture was quenched with sat. NaHCO₃, extracted with EA (20 mL), concentrated to dry, and purified by column chromatography (DCM/MeOH) to obtain Ly101-27 (220 mg, 41%). MS (ESI) m/z: 750.3 [M+Na]⁺.

### EXAMPLE 7

### Synthesis of Ly101-31

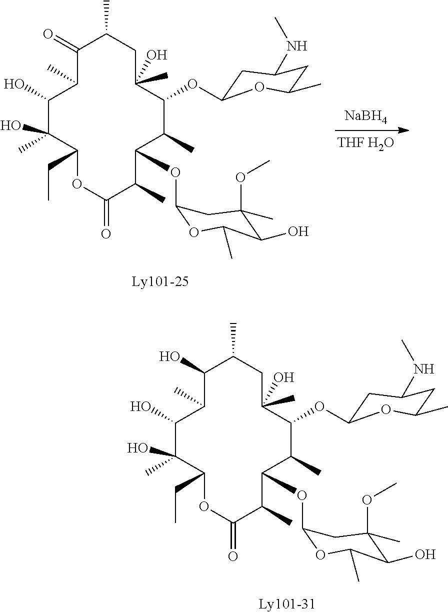

Ly101-25 (0.3 g, 0.42 mmol) was dissolved in THF (5 mL). To the solution an aqueous solution of NaBH₄ (36 mg, 0.94 mmol, 0.2 ml) was added dropwise at 0° C. within 1 min. The mixture was stirred at 0° C. for 1.5 h and at RT for another 3 h. The reaction mixture was quenched with citric acid, extracted with EA (10 mL), concentrated to dry, and purified by column chromatography (DCM/MeOH) to obtain Ly101-31 (200 mg, 67%). MS (ESI) m/z: 706.4 [M+H]+.

### EXAMPLE 8

### Synthesis of Ly101-32

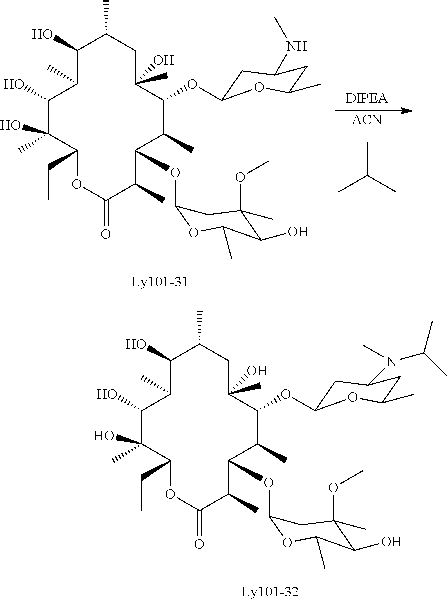

To a 50 mL single-neck flask was added Ly101-31 (0.5 g, 0.71 mmol, 1.0 eq), DIPEA (0.54 g, 4.1 mmol, 5.7 eq), iso-propyl iodide (0.47 g, 2.7 mmol, 3.9 eq). It was dissolved with ACN (10 ml). The solution was stirred at 77° C. for 15 h. TLC (DCM/MeOH/aq. Ammonia=10:100:1) showed the reaction was completed. The reaction mixture was concentrated to dry and purified by column chromatography (DCM/MeOH) to obtain Ly101-32 (230 mg, 43.3%). MS (ESI) m/z: 748.9 [M+H]⁺.

### EXAMPLE 9

### Synthesis of Ly101-33

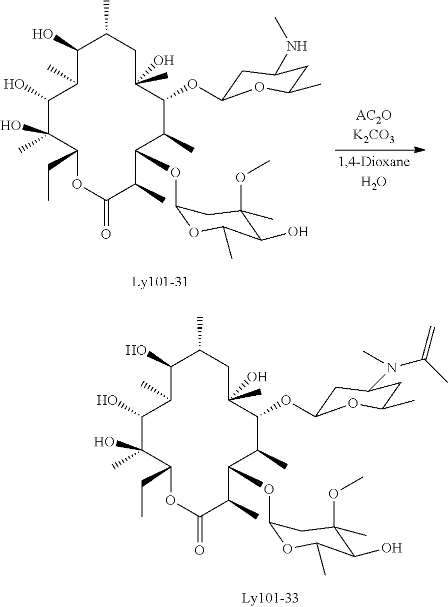 [SMILES: CC[C@H]1OC(=O)[C@H](C)[C@@H](OC2CC(C)(OC)C(O)C(C)O2)[C@H](C)[C@@H](OC2CC(NC)CC(C)O2)[C@@](C)(O)C[C@@H](C)[C@H](O)[C@H](C)[C@@H](O)[C@]1(C)O ; C=C(C)N(C)C1CC(C)OC(O[C@@H]2[C@@H](C)[C@H](OC3CC(C)(OC)C(O)C(C)O3)[C@@H](C)C(=O)O[C@H](CC)[C@@](C)(O)[C@H](O)[C@@H](C)[C@@H](O)[C@H](C)C[C@]2(C)O)C1]

To a 50 ml single-neck flask was added Ly101-31 (0.30 g, 0.42 mmol, 1.0 eq), acetic anhydride (91 mg, 0.89 mmol, 2.1 eq), K₂CO₃ (0.28 g, 2.1 mmol, 5.0 eq), dioxane (5 mL), and water (5 mL). The mixture was stirred in ice bath for 30 min, then stirred for another 3 h after removing the ice bath. TLC (EA/MeOH/aq. Ammonia=3:1:0.15) showed the reaction was completed. The reaction mixture was quenched with sat. NaHCO₃, extracted with EA (20 mL), concentrated to dry, and purified by column chromatography (DCM/MeOH) to obtain Ly101-33 (120 mg, 37.7%), MS (ESI) m/z: 770.4 [M+Na]+.

### EXAMPLE 10

### Synthesis of Ly101-34

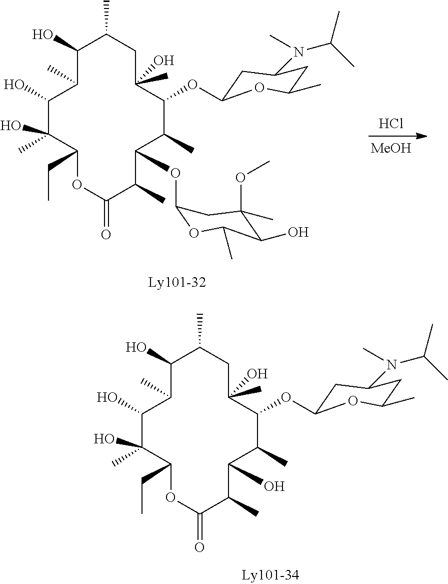 [SMILES: "MeOH" ; CC[C@H]1OC(=O)[C@H](C)[C@@H](OC2CC(C)(OC)C(O)C(C)O2)[C@H](C)[C@@H](OC2CC(N(C)C(C)C)CC(C)O2)[C@@](C)(O)C[C@@H](C)[C@H](O)[C@H](C)[C@@H](O)[C@]1(C)O ; Cl ; CC[C@H]1OC(=O)[C@H](C)[C@@H](O)[C@H](C)[C@@H](OC2CC(N(C)C(C)C)CC(C)O2)[C@@](C)(O)C[C@@H](C)[C@H](O)[C@H](C)[C@@H](O)[C@]1(C)O]

Ly101-32 (170 mg, 0.23 mmol) was dissolved in MeOH (10 ml) at RT. Conc. HCl (0.25 mL) was added dropwise to the solution and the solution was stirred at 38° C. for 2 h. The reaction was completed. The reaction mixture was adjusted to pH=7-8 using aq. ammonia, concentrated to dry, and purified by column chromatography (DCM/MeOH) to obtain Ly101-34 (84 mg, 62%). MS (ESI) m/z: 590.6 [M+H]⁺.

### EXAMPLE 11

### Synthesis of Ly101-39

Step 1: Synthesis of Ly101-38

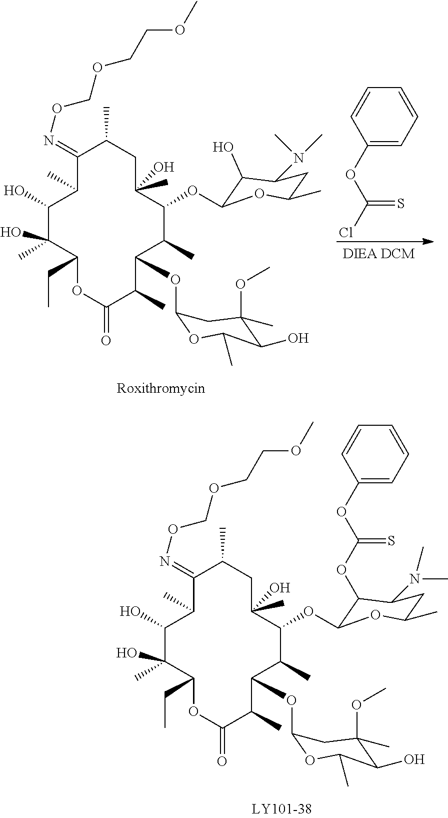

Roxithromycin (1 g, 1.2 mmol) was dissolved in dichloromethane (20 ml) and was added N,N-diisopropylethylamine (232 mg, 1.7 mmol) and phenyl thiochloroformate (309 mg, 1.7 mmol). The mixture was stirred at room temperature for 3 hours. TLC showed the reaction was completed. The reaction mixture was concentrated, purified by column chromatography (DCM/iso-propanol) to obtain Ly101-38 (587 mg, 50%). MS (ESI) m/z: 974.3 [M+H]⁺.

Step 2: Synthesis of Ly101-39

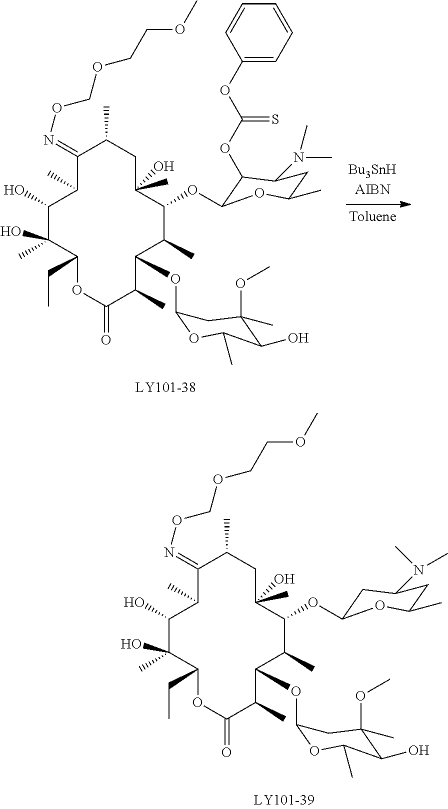 [SMILES: CC[C@H]1OC(=O)[C@H](C)[C@@H](OC2CC(C)(OC)C(O)C(C)O2)[C@H](C)[C@@H](OC2OC(C)CC(N(C)C)C2OC(=S)Oc2ccccc2)[C@@](C)(O)C[C@@H](C)/C(=N\OCOCCOC)[C@H](C)[C@@H](O)[C@]1(C)O ; CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(O)C[C@@H](C)/C(=N\OCOCCOC)[C@H](C)[C@@H](O)[C@]1(C)O]

LY101-38 (587 mg, 0.6 mmol) was dissolved in toluene (10 mL), and added AIBN (30 mg, 0.18 mmol) and tri-n-butyltin hydride (526 mg, 1.8 mmol). The mixture was stirred at 90° C. for 3 h. TLC showed the reaction was completed. The reaction mixture was concentrated, purified by column chromatography (DCM/MeOH) to obtain Ly101-39 (400 mg, 81%). MS (ESI) m/z: 821.9 [M+H]⁺.

### EXAMPLE 12

### Synthesis of Ly101-40

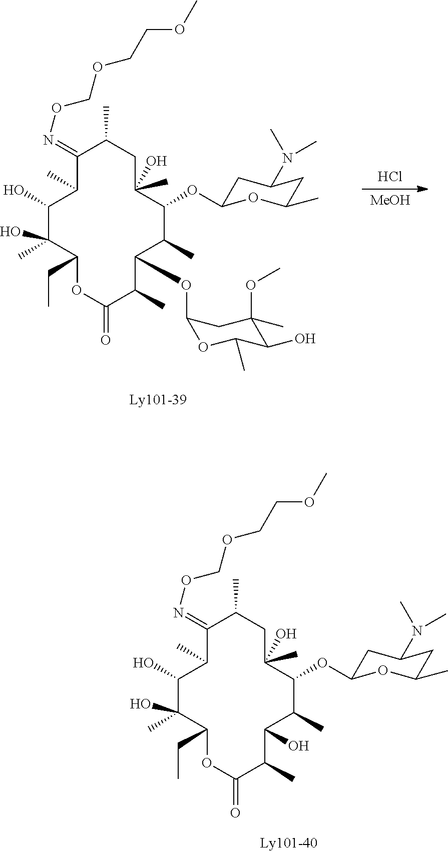 [SMILES: "MeOH" ; CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(O)C[C@@H](C)/C(=N\OCOCCOC)[C@H](C)[C@@H](O)[C@]1(C)O ; Cl ; CC[C@H]1OC(=O)[C@H](C)[C@@H](O)[C@H](C)[C@@H](OC2CC(N(C)C)CC(C)O2)[C@@](C)(O)C[C@@H](C)/C(=N\OCOCCOC)[C@H](C)[C@@H](O)[C@]1(C)O]

Ly101-39 (170 mg, 0.21 mmol) was dissolved in MeOH (10 ml) at RT, and added conc. HCl (0.25 mL). The mixture was stirred at 38° C. for 2 h. The reaction was completed. The reaction mixture was adjusted to pH=7-8 using aq. ammonia, concentrated to dry, and purified by column chromatography (DCM/MeOH) to obtain Ly101-40 (70 mg, 50%). MS (ESI) m/z: 663.3 [M+H]⁺.

### EXAMPLE 13

### Synthesis of Ly101-43

Step 1: Synthesis of Ly101-41

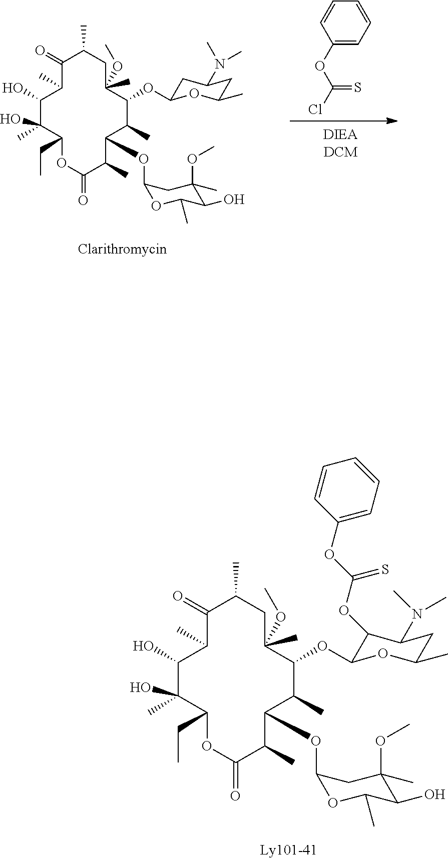 [SMILES: CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(OC)C[C@@H](C)C(=O)[C@H](C)[C@@H](O)[C@]1(C)O ; S=C(Cl)Oc1ccccc1 ; CC[C@H]1OC(=O)[C@H](C)[C@@H](OC2CC(C)(OC)C(O)C(C)O2)[C@H](C)[C@@H](OC2OC(C)CC(N(C)C)C2OC(=S)Oc2ccccc2)[C@@](C)(OC)C[C@@H](C)C(=O)[C@H](C)[C@@H](O)[C@]1(C)O]

Clarithromycin (1 g, 1.3 mmol) was dissolved in dichloromethane (20 ml), and N,N-diisopropylethylamine (258 mg, 2.0 mmol) and phenyl thiochloroformate (346 mg, 2.0 mmol) were added. The mixture was stirred at room temperature for 3 hours. TLC showed the reaction completed. The reaction mixture was concentrated, purified by column chromatography (DCM/iso-propanol) to obtain Ly101-41 (700 mg, 60%). MS (ESI) m/z: 885.1 [M+H]⁺.

Step 2: Synthesis of Ly101-43

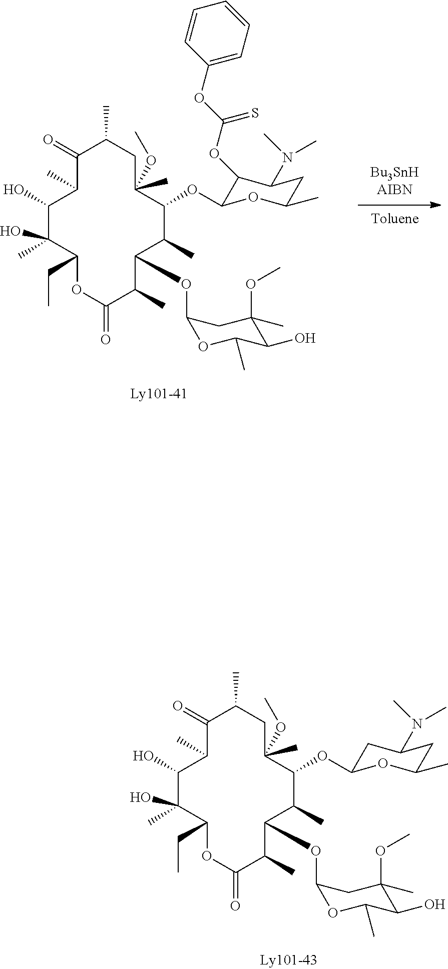 [SMILES: CC[C@H]1OC(=O)[C@H](C)[C@@H](OC2CC(C)(OC)C(O)C(C)O2)[C@H](C)[C@@H](OC2OC(C)CC(N(C)C)C2OC(=S)Oc2ccccc2)[C@@](C)(OC)C[C@@H](C)C(=O)[C@H](C)[C@@H](O)[C@]1(C)O ; CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(OC)C[C@@H](C)C(=O)[C@H](C)[C@@H](O)[C@]1(C)O]

A solution of LY101-41 (530 mg, 0.6 mmol) in toluene (10 mL) was added AIBN (30 mg, 0.18 mmol) and tri-n-butyl tin hydride (526 mg, 1.8 mmol), and stirred at 90° C. for 3 h. TLC showed the reaction completed. The reaction mixture was concentrated, purified by column chromatography (DCM/MeOH) to obtain Ly101-43 (360 mg, 81.9%). MS (ESI) m/z: 732.7 [M+H]⁺.

### EXAMPLE 14

### Synthesis of Ly101-44

Step 1: Synthesis of Ly101-42

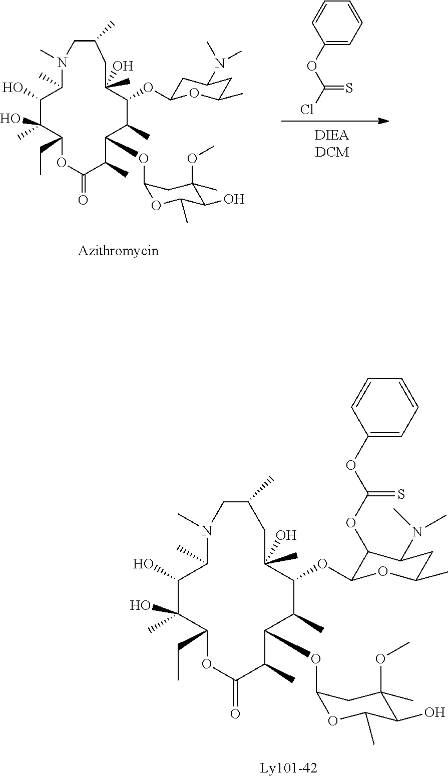 [SMILES: CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(O)C[C@@H](C)CN(C)[C@H](C)[C@@H](O)[C@]1(C)O ; S=C(Cl)Oc1ccccc1 ; CC[C@H]1OC(=O)[C@H](C)[C@@H](OC2CC(C)(OC)C(O)C(C)O2)[C@H](C)[C@@H](OC2OC(C)CC(N(C)C)C2OC(=S)Oc2ccccc2)[C@@](C)(O)C[C@@H](C)CN(C)[C@H](C)[C@@H](O)[C@]1(C)O]

Azithromycin (0.97 mg, 1.3 mmol) was dissolved in dichloromethane (20 ml), and N,N-diisopropylethylamine (258 mg, 2.0 mmol) and phenyl thiochloroformate (346 mg, 2.0 mmol) were added. The mixture was stirred at room temperature for 3 hours. TLC showed the reaction was completed. The reaction mixture was concentrated, purified by column chromatography (DCM/iso-propanol) to obtain Ly101-42 (700 mg, 60.8%). MS (ESI) m/z: 886.1 [M+H]⁺.

Step 2: Synthesis of Ly101-44

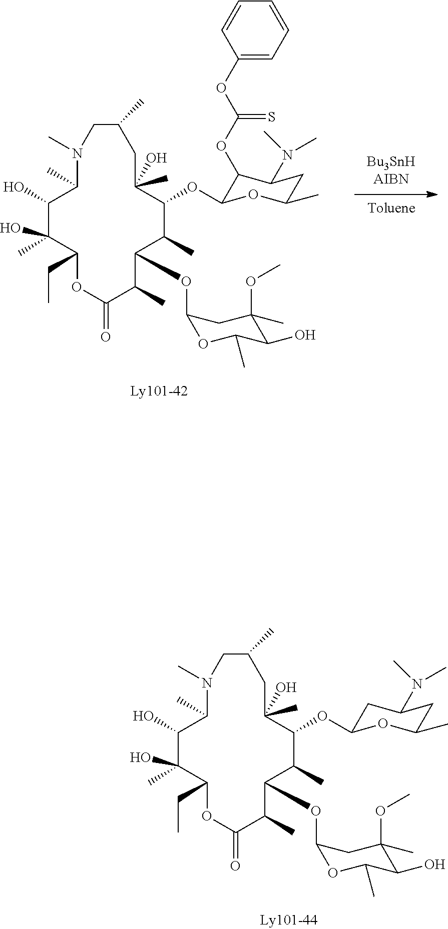 [SMILES: CC[C@H]1OC(=O)[C@H](C)[C@@H](OC2CC(C)(OC)C(O)C(C)O2)[C@H](C)[C@@H](OC2OC(C)CC(N(C)C)C2OC(=S)Oc2ccccc2)[C@@](C)(O)C[C@@H](C)CN(C)[C@H](C)[C@@H](O)[C@]1(C)O ; CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(O)C[C@@H](C)CN(C)[C@H](C)[C@@H](O)[C@]1(C)O]

To a solution of LY101-42 (531 mg, 0.6 mmol) in toluene (10 mL) was added AIBN (30 mg, 0.18 mmol) and tri-n-butyl tin hydride (526 mg, 1.8 mmol), and the solution was stirred at 90° C. for 3 h. TLC showed that the reaction was completed. The reaction mixture was concentrated, purified by column chromatography (DCM/MeOH) to obtain Ly101-44 (352 mg, 80%). MS (ESI) m/z: 733.8 [M+H]⁺.

### EXAMPLE 15

### Synthesis of Ly101-45

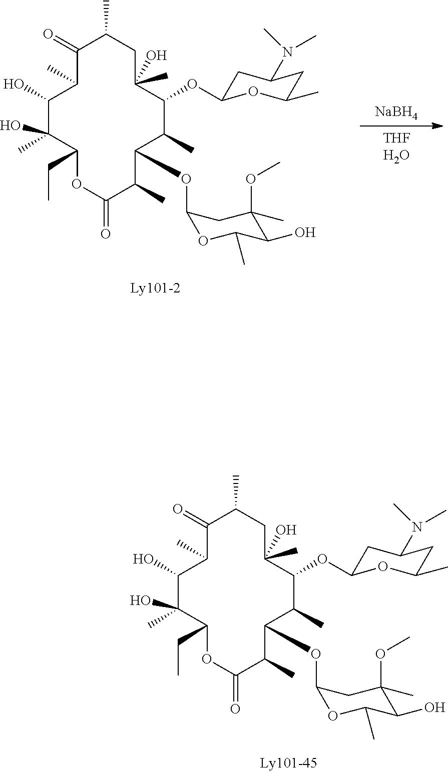 [SMILES: CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(O)C[C@@H](C)C(=O)[C@H](C)[C@@H](O)[C@]1(C)O ; "NaBH4" ; CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(O)C[C@@H](C)C(=O)[C@H](C)[C@@H](O)[C@]1(C)O]

Ly101-2 (0.3 g, 0.42 mmol) was dissolved in THF (5 mL) and an aqueous solution of NaBH₄ (36 mg, 0.94 mmol, 0.2 ml) was added dropwise at 0° C. within 1 min. The mixture was stirred at 0° C. for 1.5 h and at RT for another 3 h, and quenched with citric acid. The reaction mixture was extracted with DCM, concentrated, and purified by column chromatography (DCM/MeOH) to obtain Ly101-45 (100 mg, 33%). MS (ESI) m/z: 720.7 [M+H]⁺.

### EXAMPLE 16

### Synthesis of Ly101-46

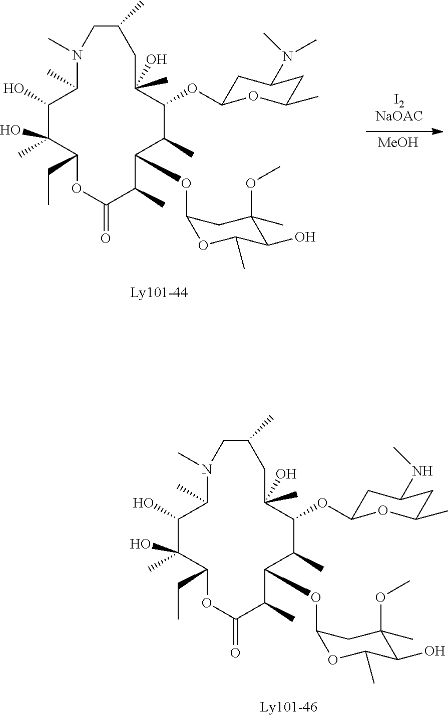 [SMILES: "MeOH" ; CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(O)C[C@@H](C)CN(C)[C@H](C)[C@@H](O)[C@]1(C)O ; CC[C@H]1OC(=O)[C@H](C)[C@@H](OC2CC(C)(OC)C(O)C(C)O2)[C@H](C)[C@@H](OC2CC(NC)CC(C)O2)[C@@](C)(O)C[C@@H](C)CN(C)[C@H](C)[C@@H](O)[C@]1(C)O]

To a 50 mL single-neck flask was added Ly101-44 (183 mg, 0.25 mmol, 1.0 eq), iodine (89 mg, 0.35 mmol, 1.4 eq), and sodium acetate (117 mg, 1.4 mmol, 5.7 eq). It was dissolved with MeOH (6 ml) and water (1 mL).The solution was stirred at 50° C., adjusted to pH=8-9 using aq. NaOH (0.5 mol/L) and heated for 2 h. TLC (DCM/MeOH/aq. Ammonia=10:100:1) showed the reaction was completed. The reaction mixture was concentrated to dry, and purified by column (DCM/MeOH) to obtain Ly101-46 (945 mg, 25%). MS (ESI) m/z: 719.7 [M+H]⁺.

### EXAMPLE 17

### Synthesis of Ly101-47

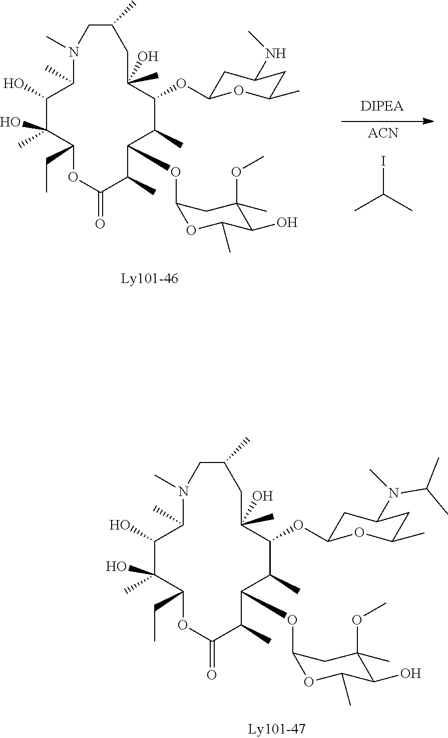

To a 50 mL single-neck flask was added Ly101-46 (0.51 g, 0.71 mmol, 1.0 eq), DIPEA (0.54 g, 4.1 mmol, 5.7 eq) and iso-propyl iodide (0.47 g, 2.7 mmol, 3.9 eq). It was dissolved with ACN (10 ml). The mixture was stirred at 77° C. for 15 h. TLC (DCM/MeOH/aq. Ammonia=10:100:1) showed the reaction was completed. The reaction mixture was concentrated to dry, purified by column chromatography (DCM/MeOH) to obtain product Ly101-47 (250 mg, 46%). MS (ESI) m/z: 761.4 [M+H]⁺.

### EXAMPLE 18

### Synthesis of Ly101-48

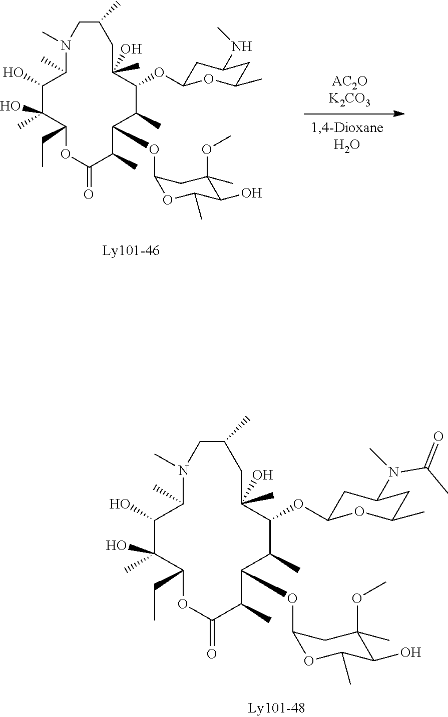 [SMILES: CC[C@H]1OC(=O)[C@H](C)[C@@H](OC2CC(C)(OC)C(O)C(C)O2)[C@H](C)[C@@H](OC2CC(NC)CC(C)O2)[C@@](C)(O)C[C@@H](C)CN(C)[C@H](C)[C@@H](O)[C@]1(C)O ; CC[C@H]1OC(=O)[C@H](C)[C@@H](OC2CC(C)(OC)C(O)C(C)O2)[C@H](C)[C@@H](OC2CC(N(C)C(C)=O)CC(C)O2)[C@@](C)(O)C[C@@H](C)CN(C)[C@H](C)[C@@H](O)[C@]1(C)O]

To a 50 ml single-neck flask was added Ly101-46 (0.53 g, 0.73 mmol, 1.0 eq), acetic anhydride (156.3 mg, 1.5 mmol, 2.1 eq), K₂CO₃ (0.5 g, 3.65 mmol, 5.0 eq), dioxane (5 mL), and water (5 mL). The mixture was stirred in ice bath for 30 min, then stirred for another 3 h after removing the ice bath. TLC (EA/MeOH/aq. Ammonia=3:1:0.15) showed the reaction was completed. The reaction mixture was quenched with sat. NaHCO₃, extracted with EA (20 mL), and purified by column chromatography (DCM/MeOH) to obtain Ly101-48 (231 mg, 41.5%). MS (ESI) m/z: 761.8 [M+H]⁺.

### EXAMPLE 19

### Synthesis of Ly101-51

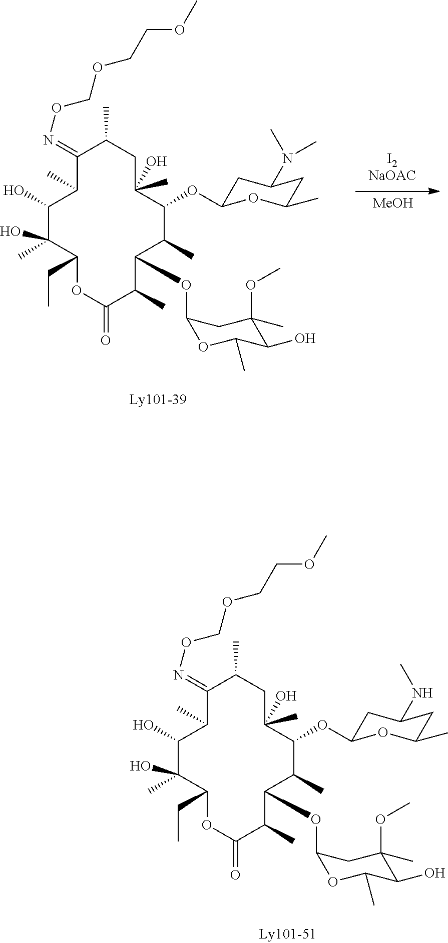 [SMILES: "MeOH" ; CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(O)C[C@@H](C)/C(=N\OCOCCOC)[C@H](C)[C@@H](O)[C@]1(C)O ; CC[C@H]1OC(=O)[C@H](C)[C@@H](OC2CC(C)(OC)C(O)C(C)O2)[C@H](C)[C@@H](OC2CC(NC)CC(C)O2)[C@@](C)(O)C[C@@H](C)/C(=N\OCOCCOC)[C@H](C)[C@@H](O)[C@]1(C)O]

To a 50 mL single-neck flask was added Ly101-39 (205 mg, 0.25 mmol, 1.0 eq), iodine (89 mg, 0.35 mmol, 1.4 eq) and sodium acetate (117 mg, 1.4 mmol, 5.7 eq). It was dissolved with MeOH (6 ml), and water (1 mL). The solution was stirred at 50° C., adjusted to pH=8-9 using aq. NaOH (0.5 mol/L) and heated for 2 h. TLC (DCM/MeOH/aq. Ammonia=10:100:1) showed the reaction was completed. The reaction mixture was concentrated to dry, and purified by column (DCM/MeOH) to obtain product Ly101-51 (45 mg, 22%).

### EXAMPLE 20

### Synthesis of Ly101-52

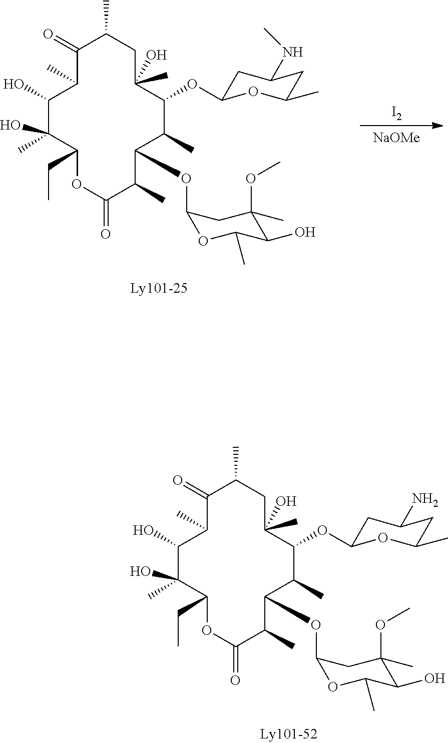 [SMILES: CC[C@H]1OC(=O)[C@H](C)[C@@H](OC2CC(C)(OC)C(O)C(C)O2)[C@H](C)[C@@H](OC2CC(NC)CC(C)O2)[C@@](C)(O)C[C@@H](C)C(=O)[C@H](C)[C@@H](O)[C@]1(C)O ; "I2" ; CC[C@H]1OC(=O)[C@H](C)[C@@H](OC2CC(C)(OC)C(O)C(C)O2)[C@H](C)[C@@H](OC2CC(N)CC(C)O2)[C@@](C)(O)C[C@@H](C)C(=O)[C@H](C)[C@@H](O)[C@]1(C)O ; "NaOMe"]

Metal sodium (48.3 mg, 2.1 mmol) was added to MeOH (15 mL) and the mixture was stirred for 30 minutes at room temperature. The mixture was cooled to 0° C. and added Ly101-25 (211.2 mg, 0.3 mmol), and iodine (380.7 mg, 1.5 mmol). The mixture was stirred at 0° C. for 5 h. TLC showed the reaction was completed. The reaction mixture was added sat. sodium thiosulphate, extracted with DCM, and purified by column chromatography (DCM/MeOH) to obtain product Ly101-52 (50 mg, 24%). MS (ESI) m/z: 690.6 [M+H]⁺.

### EXAMPLE 21

### Synthesis of Ly101-53

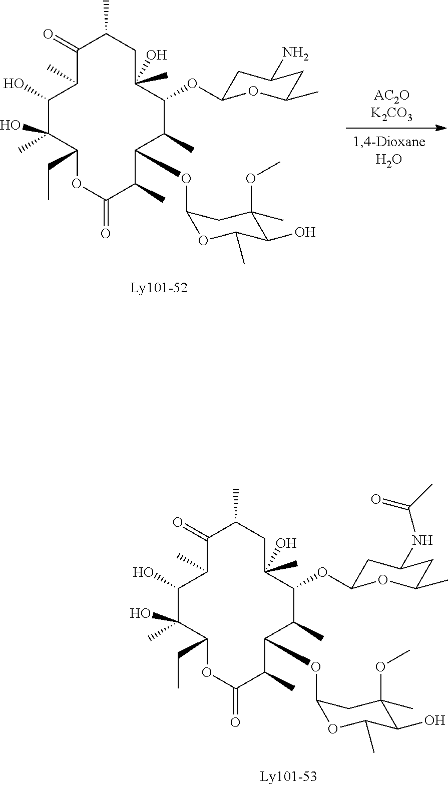 [SMILES: CC[C@H]1OC(=O)[C@H](C)[C@@H](OC2CC(C)(OC)C(O)C(C)O2)[C@H](C)[C@@H](OC2CC(N)CC(C)O2)[C@@](C)(O)C[C@@H](C)C(=O)[C@H](C)[C@@H](O)[C@]1(C)O ; CC[C@H]1OC(=O)[C@H](C)[C@@H](OC2CC(C)(OC)C(O)C(C)O2)[C@H](C)[C@@H](OC2CC(NC(C)=O)CC(C)O2)[C@@](C)(O)C[C@@H](C)C(=O)[C@H](C)[C@@H](O)[C@]1(C)O]

A 50 ml single-neck flask was added Ly101-52 (0.51 g, 0.73 mmol, 1.0 eq), acetic anhydride (156.3 mg, 1.5 mmol, 2.1 eq), K₂CO₃ (0.5 g, 3.65 mmol, 5.0 eq), and DCM (10 mL). The mixture was stirred in ice bath for 30 min, then stirred for another 3 h after removing the ice bath. TLC (EA/MeOH/aq. Ammonia=3:1:0.15) showed that the reaction was completed. The reaction mixture was quenched with sat. NaHCO₃, extracted with EA (20 mL), concentrated, and purified by column chromatography (DCM/MeOH) to obtain Ly101-53 (236 mg, 40%). MS (ESI) m/z: 755.3 [M+H]⁺.

### EXAMPLE 22

### Synthesis of Ly101-54

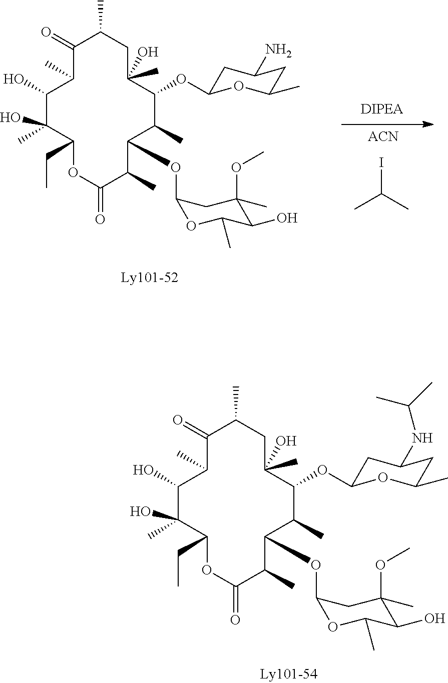

To a 50 mL single-neck flask was added Ly101-52 (0.48 g, 0.71 mmol, 1.0 eq), DIPEA (0.54 g, 4.1 mmol, 5.7 eq) and iso-propyl iodide (0.47 g, 2.7 mmol, 3.9 eq). It was dissolved with ACN (10 ml). The mixture was stirred at 77° C. for 15 h. TLC (DCM/MeOH/aq. Ammonia=10:100:1) showed that the reaction was completed. The reaction mixture was concentrated to dry, and purified by column chromatography (DCM/MeOH) to obtain product Ly101-54 (243 mg, 50.6%). MS (ESI) m/z: 732.5 [M+H]⁺.

### EXAMPLE 23-25

Other compounds in Table A were prepared in method that similar to Example 1-22 with different.

TABLE A
Compounds

| Ex. | Cmpd. | |
|---|---|---|
| No. | No. | Structure |
| 1 | Ly 101-25 | 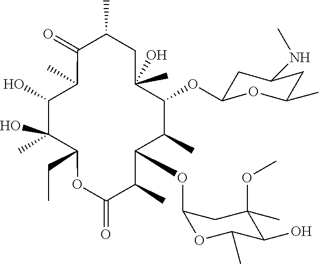 [SMILES: CC[C@H]1OC(=O)[C@H](C)[C@@H](OC2CC(C)(OC)C(O)C(C)O2)[C@H](C)[C@@H](OC2CC(NC)CC(C)O2)[C@@](C)(O)C[C@@H](C)C(=O)[C@H](C)[C@@H](O)[C@]1(C)O] |
| 2 | Ly 101-22 | 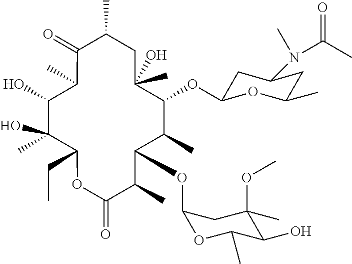 [SMILES: CC[C@H]1OC(=O)[C@H](C)[C@@H](OC2CC(C)(OC)C(O)C(C)O2)[C@H](C)[C@@H](OC2CC(N(C)C(C)=O)CC(C)O2)[C@@](C)(O)C[C@@H](C)C(=O)[C@H](C)[C@@H](O)[C@]1(C)O] |
| 3 | Ly 101-3 | 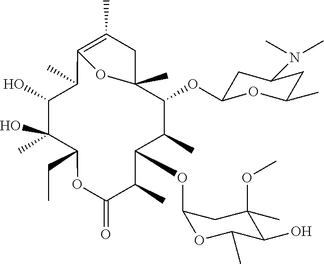 |
| 4 | Ly 101-6 | 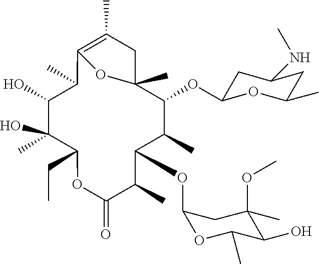 |
| 5 | Ly 101-24 | 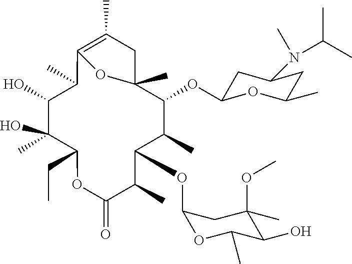 |
| 6 | Ly 101-27 | 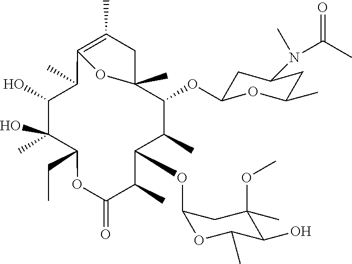 |
| 7 | Ly 101-31 | 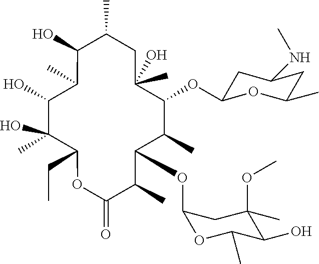 [SMILES: CC[C@H]1OC(=O)[C@H](C)[C@@H](OC2CC(C)(OC)C(O)C(C)O2)[C@H](C)[C@@H](OC2CC(NC)CC(C)O2)[C@@](C)(O)C[C@@H](C)[C@H](O)[C@H](C)[C@@H](O)[C@]1(C)O] |
| 8 | Ly 101-32 | 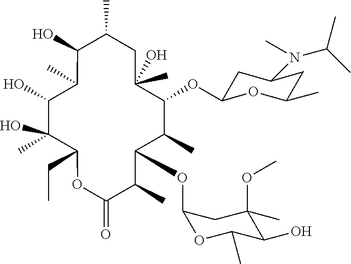 [SMILES: CC[C@H]1OC(=O)[C@H](C)[C@@H](OC2CC(C)(OC)C(O)C(C)O2)[C@H](C)[C@@H](OC2CC(N(C)C(C)C)CC(C)O2)[C@@](C)(O)C[C@@H](C)[C@H](O)[C@H](C)[C@@H](O)[C@]1(C)O] |
| 9 | Ly 101-33 | 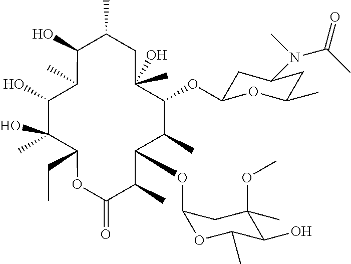 [SMILES: CC[C@H]1OC(=O)[C@H](C)[C@@H](OC2CC(C)(OC)C(O)C(C)O2)[C@H](C)[C@@H](OC2CC(N(C)C(C)=O)CC(C)O2)[C@@](C)(O)C[C@@H](C)[C@H](O)[C@H](C)[C@@H](O)[C@]1(C)O] |
| 10 | Ly 101-34 | 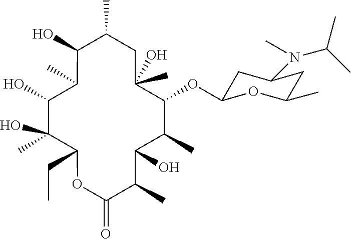 [SMILES: CC[C@H]1OC(=O)[C@H](C)[C@@H](O)[C@H](C)[C@@H](OC2CC(N(C)C(C)C)CC(C)O2)[C@@](C)(O)C[C@@H](C)[C@H](O)[C@H](C)[C@@H](O)[C@]1(C)O] |
| 11 | Ly 101-39 | 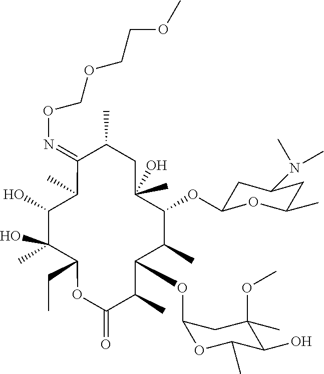 [SMILES: CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(O)C[C@@H](C)/C(=N\OCOCCOC)[C@H](C)[C@@H](O)[C@]1(C)O] |
| 12 | Ly 101-40 | 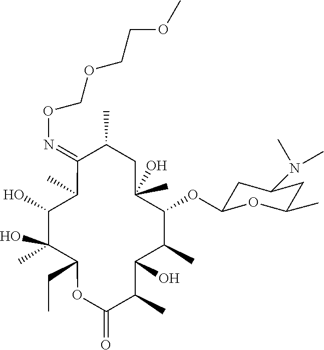 [SMILES: CC[C@H]1OC(=O)[C@H](C)[C@@H](O)[C@H](C)[C@@H](OC2CC(N(C)C)CC(C)O2)[C@@](C)(O)C[C@@H](C)/C(=N\OCOCCOC)[C@H](C)[C@@H](O)[C@]1(C)O] |
| 13 | Ly 101-43 | 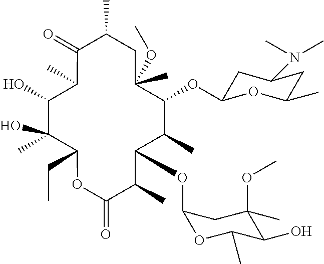 [SMILES: CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(OC)C[C@@H](C)C(=O)[C@H](C)[C@@H](O)[C@]1(C)O] |
| 14 | Ly 101-44 | 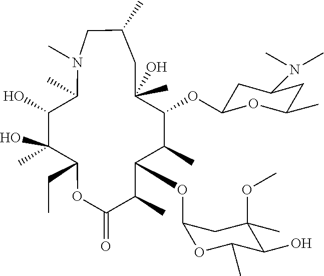 [SMILES: CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(O)C[C@@H](C)CN(C)[C@H](C)[C@@H](O)[C@]1(C)O] |
| 15 | Ly 101-45 | 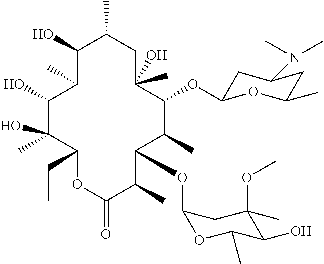 [SMILES: CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(O)C[C@@H](C)[C@H](O)[C@H](C)[C@@H](O)[C@]1(C)O] |
| 16 | Ly 101-46 | 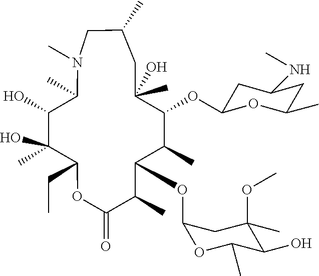 [SMILES: CC[C@H]1OC(=O)[C@H](C)[C@@H](OC2CC(C)(OC)C(O)C(C)O2)[C@H](C)[C@@H](OC2CC(NC)CC(C)O2)[C@@](C)(O)C[C@@H](C)CN(C)[C@H](C)[C@@H](O)[C@]1(C)O] |
| 17 | Ly 101-47 | 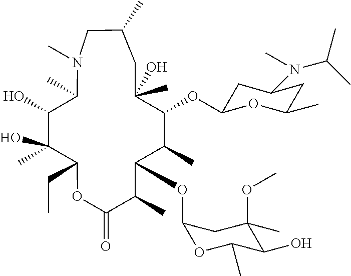 [SMILES: CC[C@H]1OC(=O)[C@H](C)[C@@H](OC2CC(C)(OC)C(O)C(C)O2)[C@H](C)[C@@H](OC2CC(N(C)C(C)C)CC(C)O2)[C@@](C)(O)C[C@@H](C)CN(C)[C@H](C)[C@@H](O)[C@]1(C)O] |
| 18 | Ly 101-48 | 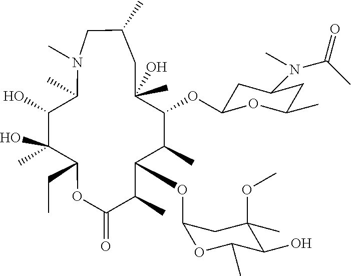 [SMILES: CC[C@H]1OC(=O)[C@H](C)[C@@H](OC2CC(C)(OC)C(O)C(C)O2)[C@H](C)[C@@H](OC2CC(N(C)C(C)=O)CC(C)O2)[C@@](C)(O)C[C@@H](C)CN(C)[C@H](C)[C@@H](O)[C@]1(C)O] |
| 19 | Ly 101-51 | 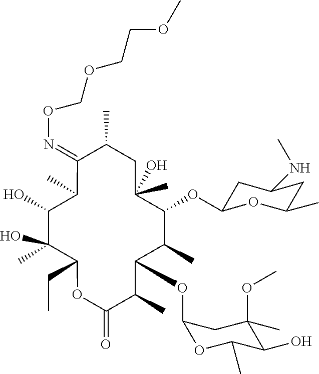 [SMILES: CC[C@H]1OC(=O)[C@H](C)[C@@H](OC2CC(C)(OC)C(O)C(C)O2)[C@H](C)[C@@H](OC2CC(NC)CC(C)O2)[C@@](C)(O)C[C@@H](C)/C(=N\OCOCCOC)[C@H](C)[C@@H](O)[C@]1(C)O] |
| 20 | Ly 101-52 | 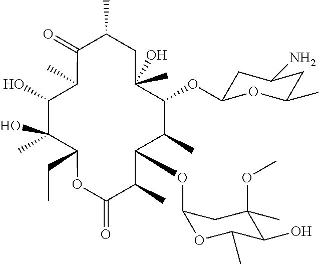 [SMILES: CC[C@H]1OC(=O)[C@H](C)[C@@H](OC2CC(C)(OC)C(O)C(C)O2)[C@H](C)[C@@H](OC2CC(N)CC(C)O2)[C@@](C)(O)C[C@@H](C)C(=O)[C@H](C)[C@@H](O)[C@]1(C)O] |
| 21 | Ly 101-53 | 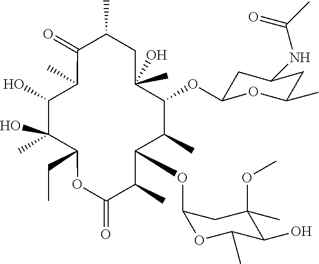 [SMILES: CC[C@H]1OC(=O)[C@H](C)[C@@H](OC2CC(C)(OC)C(O)C(C)O2)[C@H](C)[C@@H](OC2CC(NC(C)=O)CC(C)O2)[C@@](C)(O)C[C@@H](C)C(=O)[C@H](C)[C@@H](O)[C@]1(C)O] |
| 22 | Ly 101-54 | 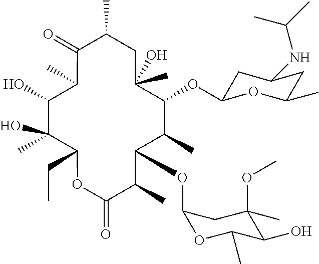 [SMILES: CC[C@H]1OC(=O)[C@H](C)[C@@H](OC2CC(C)(OC)C(O)C(C)O2)[C@H](C)[C@@H](OC2CC(NC(C)C)CC(C)O2)[C@@](C)(O)C[C@@H](C)C(=O)[C@H](C)[C@@H](O)[C@]1(C)O] |
| 23 | Ly 101-20 | 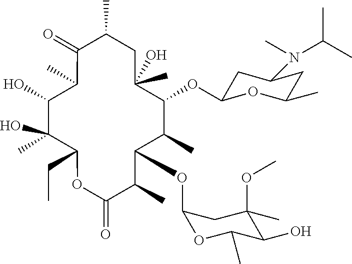 [SMILES: CC[C@H]1OC(=O)[C@H](C)[C@@H](OC2CC(C)(OC)C(O)C(C)O2)[C@H](C)[C@@H](OC2CC(N(C)C(C)C)CC(C)O2)[C@@](C)(O)C[C@@H](C)C(=O)[C@H](C)[C@@H](O)[C@]1(C)O] |
| 24 | Ly 101-26 | 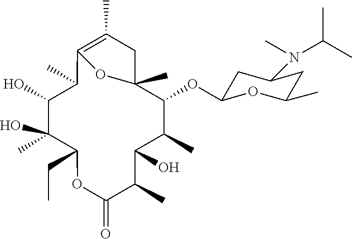 |
| 25 | Ly 101-30 | 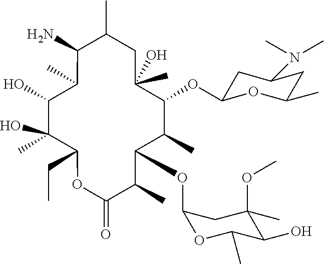 [SMILES: CC[C@H]1OC(=O)[C@H](C)[C@@H](OC2CC(C)(OC)C(O)C(C)O2)[C@H](C)[C@@H](OC2CC(N(C)C)CC(C)O2)[C@@](C)(O)CC(C)[C@H](N)[C@H](C)[C@@H](O)[C@]1(C)O] |

### EXAMPLE 26

### Synthesis of Ly101-4

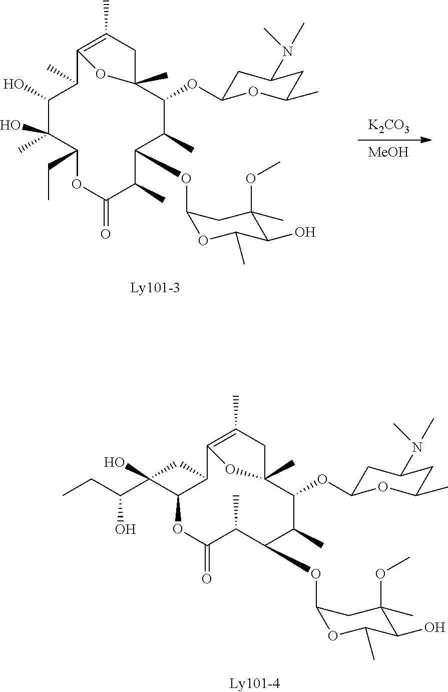

In 100 ml single-necked flask was charged with Ly101-3 (6 g, 8.5 mmol, 1.0 eq), then K₂CO₃ (0.98 g, 7 mmol, 0.8 eq). MeOH (200 mL) was added to obtain a clear solution. The solution was heated at reflux for 2 h. TLC (DCM/MeOH/aq. Ammonia=10:100:1) showed reaction completed. The reaction mixture was concentrated to solid, washed with DCM (20 ml) and sat. NaHCO₃ (10 mL), dried over anhydrous Na₂SO₄, concentrated and purified by Column (DCM/MeOH) to obtain Ly101-4 (5 g, 83%). MS (ESI) m/z: 700.4 [M+H]⁺

### EXAMPLE 27

### Synthesis of Ly101-10

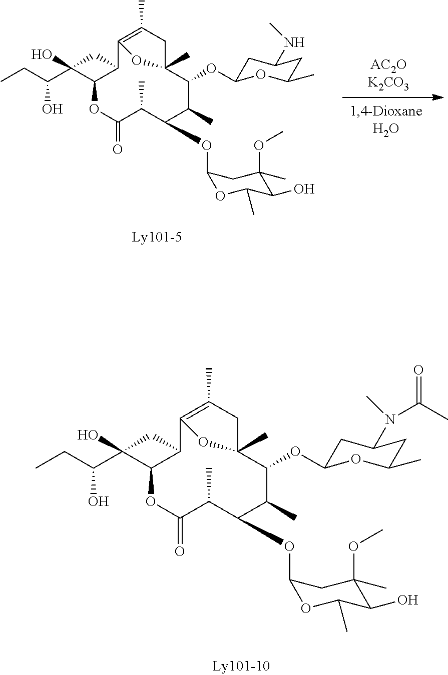

A 50 ml single-neck flask was charged with Ly101-5 (100 mg, 0.14 mmol, 1.0 eq), acetic anhydride (30 mg, 0.29 mmol, 2.1 eq), K₂CO₃ (96.6 mg, 0.7 mmol, 5.0 eq), dioxane (5 mL), and water (5 mL). The mixture was stirred in ice bath for 30 min, followed with removing the ice bath and stirred for another 3 h. TLC (EA/MeOH/aq. Ammonia=3:1:0.15) showed reaction completed. The reaction mixture was quenched with sat. NaHCO₃, extracted with EA (20 mL), concentrated, and purified by Column (DCM/MeOH) to obtain Ly101-10 (30 mg, 29.4%), MS (ESI) m/z: 750.3 [M+Na]⁺.

### Test Example 1

### Effect on Bacterial Growth

The antibacterial activity of Erythromycin and compounds of examples were evaluated by the agar dilution method, following the guidelines of the CLSI. Table 1 showed various bacteria strains tested in the assay. Briefly, bacteria were grown overnight in adapted media (MHIIA with 5% of sheep blood for Streptococcus pneumoniae and Haemophilus influenzae, and MHIIA for the other bacteria strains) at 35° C., 5% CO₂.

Cultures were centrifuged at 3000×g for 15 min. Pellets were diluted with 5 mls cold PBS and the OD₆₀₀ₙₘ was adjusted to 10⁸ CFU.mL⁻¹ with a spectrometer. Bacteria were then added to 96-well test plates. Compounds were diluted in DMSO in a 2 fold steps from 20 mg/ml in DMSO in 96 well plated and transferred to the test plate. During the incubation, the plate was maintained at 35° C., 5% CO₂.

The minimum inhibitory concentration (MIC) was defined as the lowest concentration of antibiotic that completely inhibited growth of the organism in the agar plate as detected by unaided eyes. The MICs for Erythromycin and compounds of examples were determined after 20-24 hrs incubation.

TABLE 1
Description of bacteria strains used in the antibacterial activity assay and summary of the MIC of
erythromycin and Compounds of examples

| | | | minimum inhibitory concentration | | | | | |
|---|---|---|---|---|---|---|---|---|
| | | | MIC (μg/ml) | | | | | |
| Bacteria | | | | LY101- | LY101- | LY101- | LY101- | LY101- |
| Strain | Name | Supplier | Erythromycin | 2 | 22 | 25 | 39 | 45 |
| Gram | Enterococcus | ATCC | 1 | >128 | >128 | >128 | >128 | >128 |
| positive | faecalis | 29212 | | | | | | |
| bacteria | Staphylococcus | ATCC | 0.5 | 64 | >128 | >128 | >128 | >128 |
| | aureus | 29213 | | | | | | |
| | Staphylococcus | NRS 384 | 64 | >128 | >128 | >128 | >128 | >128 |
| | aureus | | | | | | | |
| | Streptococcus | ATCC 6301 | <0.125 | 64 | >128 | >128 | >128 | >128 |
| | pneumoniae | | | | | | | |
| | Streptococcus | ATCC | <0.125 | 16 | >128 | >128 | >128 | >128 |
| | pneumoniae | 49619 | | | | | | |
| | Streptococcus | ATCC | <0.125 | 64 | >128 | >128 | >128 | >128 |
| | pyogenes | BAA-572 | | | | | | |
| Non- | Acinetobacter | ATCC | 64 | >128 | >128 | >128 | >128 | >128 |
| bacteria | baumanii | 17978 | | | | | | |
| fermenting | Acinetobacter | ATCC- | 16 | >128 | >128 | >128 | >128 | >128 |
| | baumannii | BAA-1605 | | | | | | |
| / | Haemophilus | ATCC | 16 | >128 | >128 | >128 | >128 | >128 |
| | influenzae | 49247 | | | | | | |
| / | Moraxella | ATCC | 2 | >128 | >128 | >128 | >128 | >128 |
| | catarrhalis | 43617 | | | | | | |

Table 1 shows that compounds of the present invention did not show any antibacterial activity.

### Test Example 2

The THP-1 cell line was purchased from ATCC (American Type Culture Collection, Manassas, Va.). Cells were maintained in growth medium (RPMI-1640) with supplemented with 10% heat inactivated bovine serum, 100× Glutamax medium and 0.05 mM β-Mercaptoethanol at 37° C. under 5% CO₂. Compounds were dissolved in 0.1% DMSO. Erythromycin was used as a positive control.

For the assessment of the effect of compounds of the present invention on differentiation of monocyte to macrophage in THP-1 cell line, cells were collected and centrifuged at 900 rpm for 4 minutes. The cell density was adjusted to 2.5×10⁵/mL. cell suspension of 400 μL was added to each well of 48-well plate following the plate layout. PMA 1 mg, a compound can induce monocyte differentiation, was dissolved in 10 mL DMSO to form 100 μg/mL solution and aliquote it into 1 mL per vial. 1 mL solution can be further aliquoted into 10 μL per vial and stored the aliquots at −20° C. Make 10-fold dilution of PMA with DMSO and then 500-fold dilution with complete medium. 50 μL solution were then added to each well of 48-well plate following the plate layout. Erythromycin and compounds of examples at stock concentration of 10 mM was serially diluted at 10-fold in DMSO. 50 μL solution was added to each well of 48-well plate following the plate layout. After 96 hrs incubation at 37° C., 5% CO₂, the plate was washed with DPBS for three times to remove non-adherent cells for three times. Complete medium 180 μL and alarmar blue 20 μL was added to each well. The fluorescent intensity was read at excitation 530 nm and emission 590 nm using PerkinElmer Victor3 after 3 hours plate incubation.

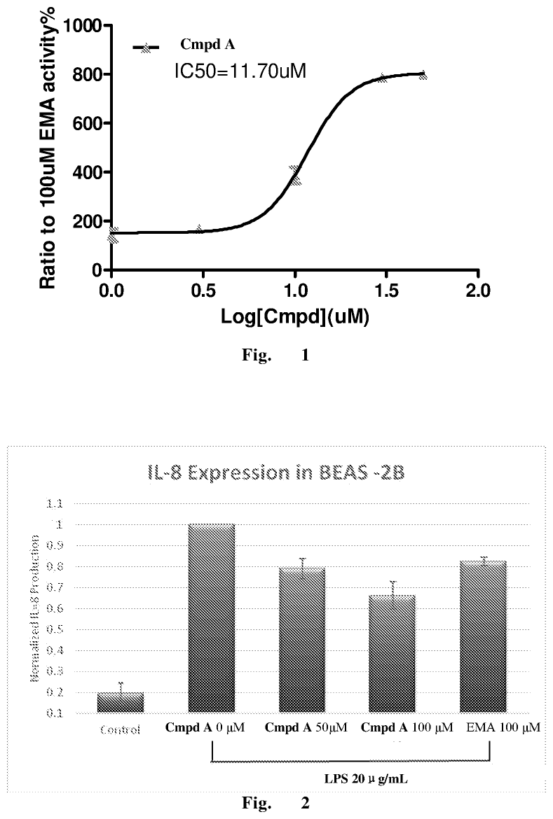

Both erythromycin and compounds of the present invention showed a promotional effect on THP-1 cell differentiation in a dose dependent manner. The differentiation activity of compounds of Examples is compared to that of 100 μM erythromycin and the results are shown in FIG. 1 and Table 3.

The medium lethal dose (LD₅₀) is the dosage of a compound that reduces the cell viability by 50%, and is determined using non-linear logistic regression.

Therapeutic window (TW) is the dosage range of a drug which can treat disease effectively without having toxic effect. TW=EC₅₀/LD₅₀.

TABLE 3
THP-1 differentiate to macrophage

| | EC50 | LD50 | Therapeutic | Maximal |
|---|---|---|---|---|
| | μM | μM | window | activation level |
| LY101-22 | 12.1 ± 2.7 | 125 ± 7.1 | ~10.3 | 7.9 ± 0.5 |
| LY101-25 | 1.0 ± 0.3 | 21.1 ± 5.3 | ~21.1 | 5.4 ± 1.1 |
| LY101-45 | 1.5 ± 0.7 | 24.2 ± 5.1 | ~16.1 | 5.3 ± 0.6 |
| LY101-39 | 10.1 ± 2.2 | 90.0 ± 18.4 | ~8.9 | 7.4 ± 0.8 |
| LY101-24 | 6.8 ± 1.4 | 56.9 ± 14.0 | ~8.4 | 7.0 ± 1.6 |
| LY101-47 | 4.3 ± 1.8 | 64.7 ± 17.9 | ~15.0 | 3.1 ± 1.1 |
| LY101-44 | 1.2 ± 0.2 | 20.4 ± 8.4 | ~17.0 | 4.5 ± 0.9 |
| Ly101-33 | 22.6 ± 5.7 | 110.0 ± 12.1 | ~4.9 | 3.14 ± 0.01 |
| LY101-27 | 12.2 ± 4.6 | 121.1 ± 7.8 | ~9.9 | 5.3 ± 1.5 |
| LY101-2 | 0.3 ± 0.03 | 4.6 ± 1.3 | ~15.3 | 6.5 ± 1.7 |
| LY101-6 | 11.5 ± 3.2 | 103.2 ± 11.4 | ~9.0 | 3.9 ± 0.8 |
| LY101-8 | 27.4 ± 7.4 | 63.7 ± 16.0 | ~2.3 | 4.0 ± 0.4 |
| LY101-48 | 18.5 ± 4.2 | 111.0 ± 13.6 | ~6.0 | 4.2 ± 1.1 |
| LY101-31 | 32.8 ± 2.5 | 115.0 ± 10.4 | ~3.5 | 6.2 ± 1.5 |
| LY101-30 | 36.8 ± 20.1 | 65.3 ± 7.1 | ~1.8 | 6.1 ± 1.2 |
| LY101-40 | 105.8 | 100 | 1 | 1.8 |
| LY101-4 | — | — | — | <1 |
| LY101-10 | — | — | — | <1 |

* Maximum activation level indicates that the ratio of the best anti-inflammatory effect of compounds tested compared to that of erythromycin 100 uM; <1 means that the anti-inflammatory effect is not as good as 100 μM erythromycin.

The result showed that compounds of the present invention promoted monocyte to macrophage differentiation in vitro.

### Test Example 3

The primary function of the bronchial epithelium is to act as a defensive barrier aiding the maintenance of normal airway function. Bronchial epithelial cells (BECs) form the interface between the external environment and the internal milieu, making it a major target of inhaled insults. BEC can also serve as effectors to initiate and orchestrate immune and inflammatory responses by releasing chemokines and cytokines, which recruit and activate inflammatory cells. By measure the secretion of cytokines like NF-κB, IL-6 and IL-8 etc., the anti-inflammatory effect of different macrolide derivatives was evaluated.

The inhibitory effect of compounds of Examples on the IL-8 expression in BEAS-2B cell was tested using the following method. BEAS-2B was purchased from ATCC (American Type Culture Collection, Manassas, Va.). Cells were maintained in growth medium (LHC-9) and cultured at 37° C. under 5% CO₂. Compounds were dissolved in 0.1% DMSO. Erythromycin was used as a positive control.

For the assessment of the effect of compounds of Examples on IL-8 expression inhibition in BEAS-2B cell line, cells were plated in 24 well plate at a density of 100,000/mL, in a final volume of 1 mL of assay medium. Cells were incubated at 37° C., 5% CO₂ for 1 day. After the incubation, compounds were added at day 2 and LPS was added at day 3. A compound source plate was prepared by triplicate five-point 10-fold or 2-fold serial dilutions in DMSO, beginning at 1 mM (final top concentration of compounds of Examples in the assay was 100 μM and the DMSO was 0.1%). The positive control consisted of cells treated with 100 μM erythromycin and negative control was consisted of 0.1% DMSO treated cells. LPS stock solution at 5 mg/mL was made by dissolving 10 mg of LPS powder into 2 mL of ddH₂O and aliquoted to 100 μL per vial. Final concentration of LPS in the assay was 20 μg/mL by making 125-fold dilution of stock solution with medium.

Specific immunoreactivity for IL-8 in culture supernatants were measured by a commercially available ELISA kits (R&D Systems, Inc., Minneapolis, Minn.). Each sample was assayed in duplicates as recommended by the manufacture. The concentration of compounds of Examples inhibiting IL-8 production on BEAS-2B by 10% was estimated from a 4-parametric of the normalized dose response curve. (IC10)

Analysis of IL-8 released by BEAS into the culture medium, demonstrated that this was significantly increased by the treatment of cells with LPS. The presence of erythromycin or compounds of Examples in the culture medium significantly inhibited LPS-induced IL-8 release. LY101-22 showed a more potent inhibitory activity than erythromycin at 100 μM concentration. (see FIG. 2 and Table 4).

The lethal dose (LD₁₀) is the dosage of a compound that reduces the cell viability by 90%, and is estimated from non-linear logistic regression.

TABLE 4
IL-8 expression in BEAS-2B cells

| | IC10 | LD10 | Therapeutic | Maximum |
|---|---|---|---|---|
| | μM | μM | window | Inhibition |
| LY101-22 | 10 | 100 | 10 | 33% |
| LY101-25 | 1 | 15 | 15 | 53% |
| LY101-45 | 1 | 15 | 15 | 40% |
| LY101-39 | 20 | 100 | 5 | 35% |
| LY101-24 | 10 | 50 | 5 | 42% |
| LY101-27 | 10 | 100 | 10 | 40% |
| LY101-33 | 20 | 60 | 3 | 25% |

FIG. 2 and Table 4 shows that compounds of the present invention inhibited IL-8 expression in

BEAS-2B cell in vitro.

### Test Example 4

Eight-week-old male C57BL/6 J mice (purchased from Shanghai Xipuer Yibikai animal Co) were randomly assigned into six groups: Control group, which intratracheally instilled with saline (50 μl); Emphysema group, which was received porcine pancreatic elastase (PPE, Sigma Chemical Co., St. Louis, Mo., USA) (0.1 UI in 50 μl saline solution) via the same route; Emphysema+Compound A groups: which received PPE intratracheally and low/mid/high dosage of Compound A orally; Emphysema+erythromycin (EM) group: which received PPE intratracheally and erythromycin 100 mg/ml orally. Saline and PPE were injected intratracheally once a week for 4 weeks. Compound A and erythromycin was given twice a day for 4 weeks. After 4 weeks, all mice were sacrificed by intraperitoneal injection of 10% chloral hydrate. Compound A is a compound selected from LY101-25, LY101-22, LY101-45, LY101-39, LY101-33, LY101-27 and LY101-48. The lung tissues were inflated with 4% paraformaldehyde at a pressure of 25 cmH₂O, fixed for 24 h in formalin, embedded with paraffin, sectioned in the sagittal plane, and stained with hematoxylin and eosin (H & E). Emphysema was quantified by measurement of the mean chord length of alveoli with Analysis software Image J.

Results:

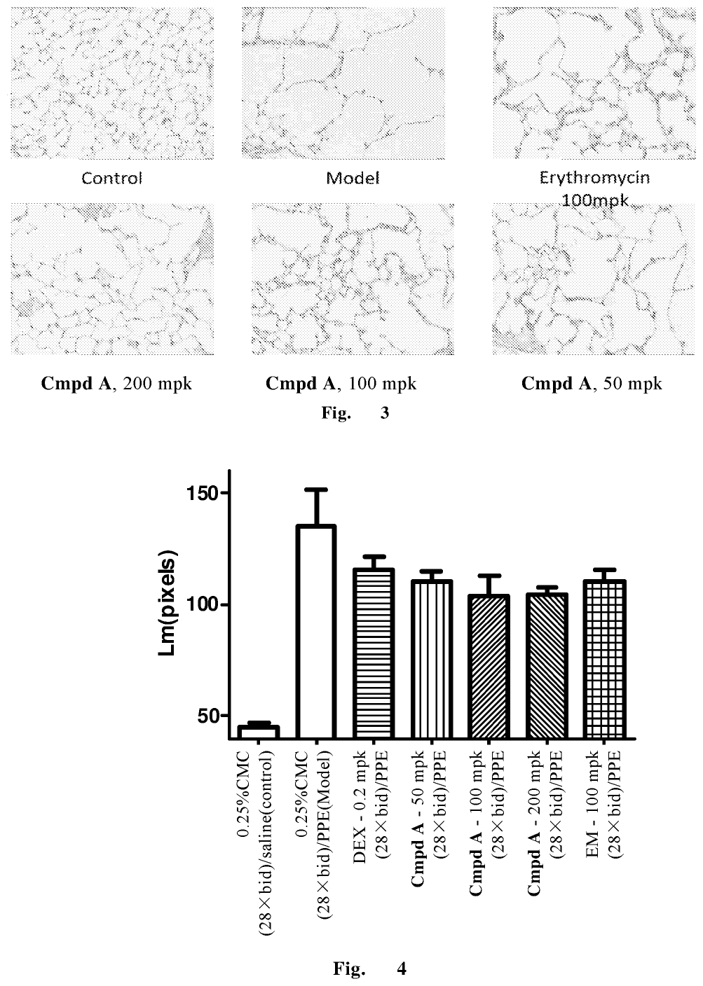

Compound A treatment reduced elastase induced emphysema. Mice instilled with elastase had diffuse emphysema lesions, and their mean alveolar chord length was markedly increased compared with that of mice instilled with saline. (FIGS. 3&4). Treatment of Compound A improved lung morphology and reduced mean chord length in a dose dependent manner. A maximum of 26% reduction in mean chord length was reached at dosage of 100 mg/ml.

The result showed that Compound A treatment significantly improved lung pathology in elastase induced emphysema mice model.

### Test Example 5

The commercial non-filtered cigarettes containing 11 mg tar and 0.9 mg nicotine per cigarette were used in this study. Eight-week-old male C57BL/6 J mice (purchased from Shanghai Xipuer Yibikai animal Co) were divided into four groups as follows: Group 1 was the control group(NS6m), Group 2 was the animal model CS group(CS6m), Groups 3 was CS+Compound A 100 mpk group(LY100), Group 4 was CS+erythromycin group(EM100). Mice were placed in a Plexiglass chamber covered by a disposable filter. The animals received CS of 5 cigarettes/time, carried out twice per day and 5 days a week for 24 weeks. Mainstream CS was generated by an exposure system in which combustion of the cigarette was drawn into the mouse chambers via a peristaltic pump. In group 3 and 4, mice were orally administrated with Compound A (100 mg/kg) twice per day from the 12th week to 24th week. One week after the last CS exposure, animals were sacrificed by intraperitoneal injection of 10% chloral hydrate for the plasma, bronchoalveolar lavage fluid (BALF) and lung tissues collection.

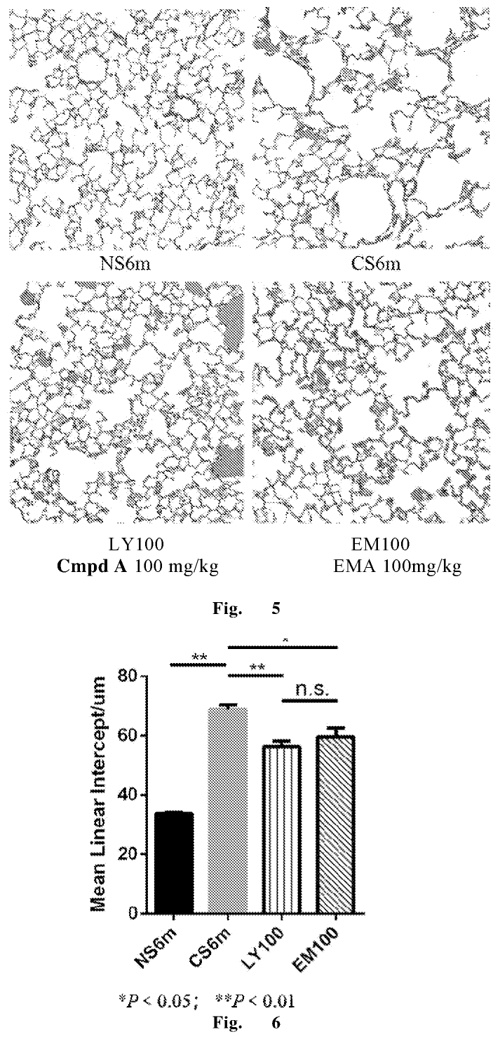

The lung tissues were collected and inflated with 4% paraformaldehyde at a pressure of 25 cmH₂O, fixed for 24 h in formalin, embedded with paraffin, sectioned in the sagittal plane, and stained with hematoxylin and eosin (H & E). Alveolus enlargement was quantified by measurement of the mean chord length of alveoli with Analysis software Image J (see FIGS. 5 & 6).

For the bronchoalveolar lavage fluid collection, lungs were lavaged with 1 ml of saline, and the resulting BALF was centrifuged at 3000 g for 15 minutes. Cells were washed three times and then analyzed on a ThermoFisher Countess II cytometer.

Results:

Compound A prevented airway histopathological changes in CS-induced COPD mice. Histological analysis of lung sections indicated that compared to the control mice, the CS group presented more inflammatory cell infiltration and enlargement of alveolus. Such changes were significantly attenuated by treatment of Compound A and erythromycin.

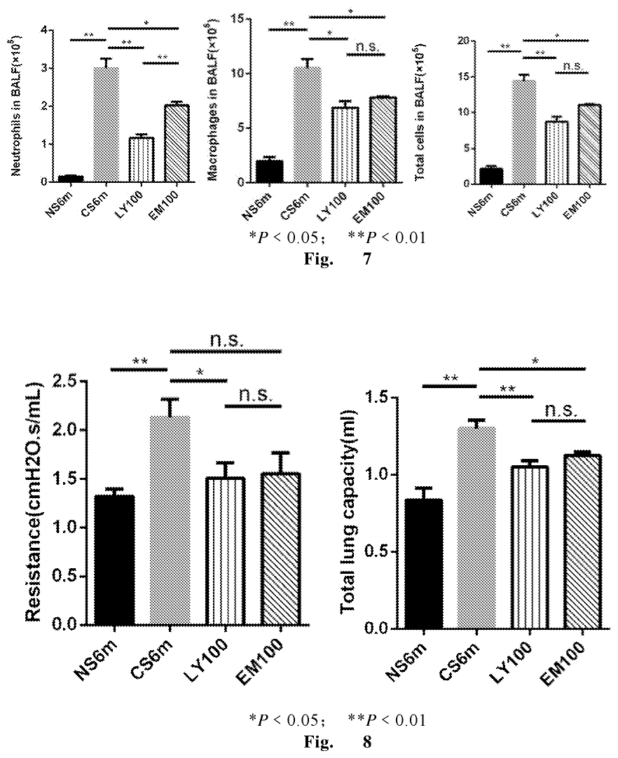

Compound A ameliorated inflammatory cell increase in BALF of CS-Induced COPD. Total cell count and the number of macrophages and neutrophils were significantly lower in the BALF of mice in the Compound A/erythromycin+smoking group compared to the smoking group. Compound A showed higher potency than erythromycin at same dosage. (FIG. 7)

Compound A partially recovered lung function in CS-induced COPD mice. Airway resistance and total lung capacity were increased after 24 weeks smoke exposure. These lung function change likely resulted from the combination of chronic inflammation, airway remodeling and emphysematous lesions with associated reductions in alveolar tissue and supporting airway attachment. Oral administration of 100 mg/kg erythromycin or Compound A reduced two parameters and partially recovered lung function. Compound A showed better therapeutic efficacy than erythromycin at same dosage. (FIG. 8)

The results showed that Compound A improved pathology and lung function in smoke induced COPD mice model.

### Test Example 6

### Acute Toxicity

The acute toxicity testing was done using the fixed dose method, following OECD guidelines 423. Briefly, the study was conducted as fixed doses of 5, 50, 300 and 2000 mg/kg using three animals from one sex in each group. A final dose is selected and three animals from the other sex are then tested. Animal Macroscopic and microscopic pathology are determined. Behavioral, biochemical parameters and mortality are also recorded.

The result showed that the acute toxicity for Compound A is quite low. For mice the oral LD 50 is higher than 2000 mg/kg body weight, and Compound A is even less toxic than erythromycin.

### Test Example 7

### Oral Bioavailability and Pharmacokenetics

A single dose of 100 mg/kg of Erythromycin or Compound A was given orally to male Sprague-Dawley rats (N-=3 per group). In another study, a single intravenous dose of two compounds was given to rats (N=3) at 30 mg/ml dosage following administration through the lateral tail vein in order to obtain the absolute oral bioavailability and clearance parameters. Compounds (10 mg/mL) was dissolved in 30% DMSO for IV injection and suspended in 0.5% CMC-Na for IG respectively. Blood samples were collected at 0.083, 0.25, 0.5, 1, 2, 4, 8, 12 and 24 h after the intravenous (i.v.) administration of erythromycin or Compound A and at 0.25, 0.5, 1, 2, 4, 6, 8, 12 and 24 h after the oral (i.g.) administration of erythromycin or Compound A. The concentrations of two compounds in plasma were determined by partially validated LC-MS/MS method.

The results showed that the clearance rates for erythromycin (EMA) and Compound A are 50.2 and 26.6 mL·kg-1·min-1 respectively. The exposure level of Compound A is twice as the level of erythromycin. The bioavailability is similar between two compounds. (see Table 2)

TABLE 2
Pharmacokenetic parameters of Compound A and Erythromycin
i.g.-100 mg/kg

| PK | | | Compound |
|---|---|---|---|
| Parameters | Unit | EMA | A |
| T_{1/2} | h | 0.930 | 0.864 |
| Tₘₐₓ | h | 2.00 | 1.50 |
| Cₘₐₓ | ng · mL⁻¹ | 2530 | 4110 |
| AUC₀₋ₜ | ng · h · mL⁻¹ | 7240 | 12400 |
| AUC_{0-inf} | ng · h · mL⁻¹ | 7250 | 12500 |
| F | % | 21.8 | 19.9 |

All literatures mentioned in the present application are incorporated herein by reference, as though each one is individually incorporated by reference. Additionally, it should be understood that after reading the above teachings, those skilled in the art can make various changes and modifications to the present invention. These equivalents also fall within the scope defined by the appended claims.

1. A compound of formula I, or a pharmaceutically acceptable salt, a stereoisomer thereof; 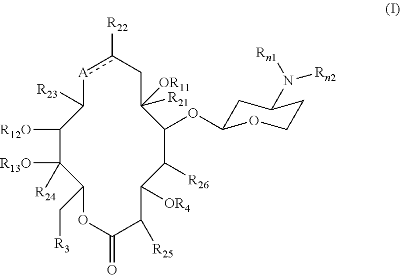
Wherein,
Rₙ₁ is selected from the group consisting of H, C₁₋₆ alkyl (preferably methyl);
Rn2 is a substituted or unsubstituted group selected from the group consisting of H, C₁₋₁₀ alkyl, —C₁-4 alkylene-C₆₋₁₀ aryl, —C₁₋₄ alkylene- (5- to 10-membered heteroaryl), C1-6 alkanoyl (C₁-6 alkyl-C(O)—), —C₁₋₆ alkanoyl-C₆₋₁₀ aryl, —C₁₋₆alkanoyl- (5- to 10-membered heteroaryl) C₁₋₆ alkoxycarbonyl (C₁₋₆ alkyl-OC (O)—), —C₁₋₆ alkoxycarbonyl-C₆₋₁₀ aryl, —C₁₋₆alkoxycarbonyl- (5- to 10-membered heteroaryl), C₂₋₁₀ alkenyl, and C₂₋₁₀ alkynyl;
R₁₂ and R₁₃ are independently selected from the group consisting of H, substituted or unsubstituted C₁₋₆ alkyl, substituted or unsubstituted C₃₋₆ cycloalkyl, R5—C(O)—, and R5—OC(O)—;
R21, R22, R23, R24, R25 and R26 are independently selected from the group consisting of H, and Substituted or unsubstituted C₁₋₆ alkyl (preferably methyl);
R₄ is selected from the group consisting of: H, substituted or unsubstituted C₁₋₆ alkyl, substituted or unsubstituted C₃₋₆ cycloalkyl, R5—C(O)—, R₅—OC(O)—, and 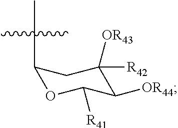
wherein,
R₄₁ and R₄₂ are independently selected from the group consisting of: H, substituted or unsubstituted C₁₋₆ alkyl;
R₄₃ is selected from the group consisting of: H, substituted or unsubstituted C₁₋₆ alkyl, Substituted or unsubstituted C₁₋₆ alkanoyl;
R₄₄ is selected from the group consisting of: H, substituted or unsubstituted C₁₋₆ alkyl, Substituted or unsubstituted C₁₋₆ alkanoyl; [symbol]
is a double bond or single bond;
R₁₁ is selected from the group consisting of H, substituted or unsubstituted C₁₋₆ alkyl, substituted or unsubstituted C₃₋₆ cycloalkyl, R₅—C(O)—, and R₅—OC(O)—; or R₁₁ is null, and when R₁₁ is null, a single bond is formed between O to which R₁₁ attached and A;
A is selected from the group consisting of —C(O)—, —N(R₆)—(C(R′)₂)—, —CR′(R₇)—, —C(═N(ORs))—, 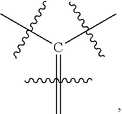 [SMILES: *C(*)=*]
—CR′═, —CR′—;
R₆ is selected from the group consisting of H, Substituted or unsubstituted C₁₋₆ alkyl;
R₇ is selected from the group consisting of: H, —OH, Substituted or unsubstituted C₁₋₆ alkyl, Substituted or unsubstituted C₁₋₆ alkoxy, Substituted or unsubstituted C₁₋₆ alkyl-C(O)O—, Substituted or unsubstituted —N (R′)₂;
R₈ is selected from the group consisting of H, —C₁₋₆ alkyl, —C₁₋₄ alkylene-C2-C6alkenyl, —C₁₋₄ alkylene-C2-C6 alkynyl, —C₁₋₄ alkylene-O—C₁₋₆ alkyl, —C₁₋₄ alkylene-S—C₁₋₆ alkyl, —C₁₋₄ alkylene-O—C₁₋₄ alkylene-O—C₁₋₆ alkyl; wherein R₈ can further be optionally substituted with a substituent selected from the group consisting of —OH, —CN, substituted or unsubstituted C₁₋₆ alkyl, substituted or unsubstituted C₆₋₁₀ aryl, substituted or unsubstituted ₅- to 10-membered heteroaryl, substituted or unsubstituted —N(R′)₂, substituted or unsubstituted C₅₋₇ heterocycloalkyl, substituted or unsubstituted C₃₋₈ cycloalkyl;
R₅ is selected from the group consisting of: H, substituted or unsubstituted C₁₋₆ alkyl, substituted or unsubstituted —C₁₋₆ alkylene-C₆₋₁₀ aryl, substituted or unsubstituted ₅- to 10-membered heteroaryl;
R′ is selected from the group consisting of: H, substituted or unsubstituted C₁₋₆ alkyl;
unless otherwise specified, the term “substituted” refers to one or more (preferably 1, 2, 3, 4 or 5) hydrogen in the group is substituted with a substituent selected from the group consisting of D, halogen, —OH, C₁₋₆ alkyl, C₁₋₆ haloalkyl.

2. The compound of claim 1, wherein Rₙ₁ is H or methyl.

3. The compound of claim 1, wherein Rₙ₂ is selected from the group consisting of: H, C₁₋₁₀ alkyl, and C₁₋₆ alkanoyl.

4. The compound of claim 1, wherein the compound of formula I has a structure of formula I-i; 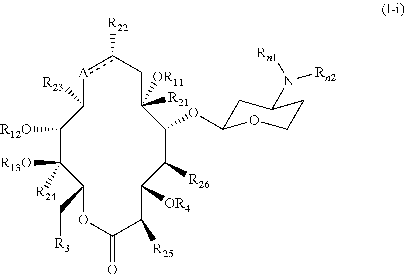
Wherein, [symbol] , Rₙ₁, Rₙ₂, R₁₁, R₁₂, R₁₃, R₂₁, R₂₂, R₂₃, R₂₄, R₂₅, R₂₆, R₃, R₄, and A are defined as above.

5. The compound of claim 1, wherein the compound of formula I has a structure of formula Ia, Ib, Ic, Id or Ie, 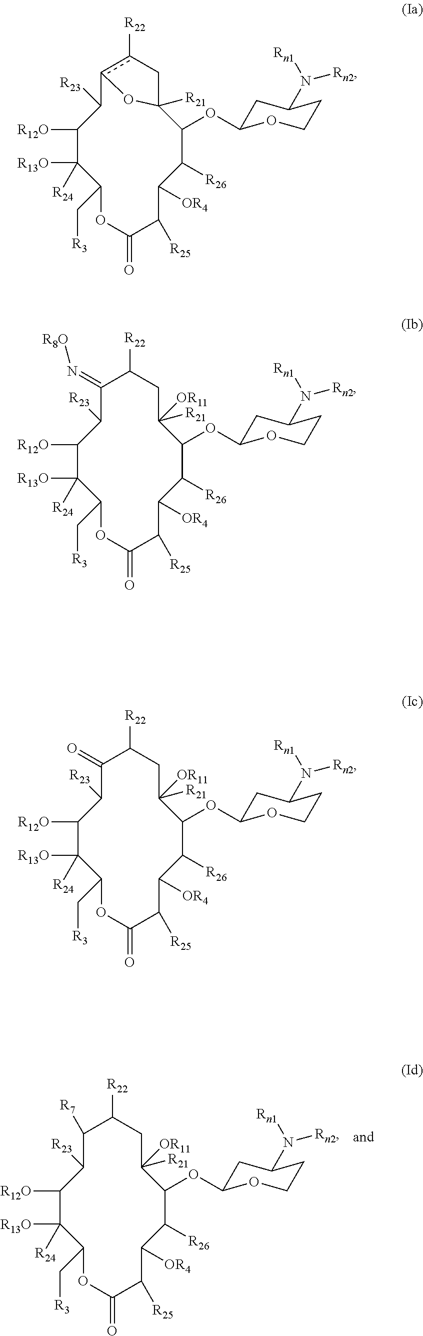 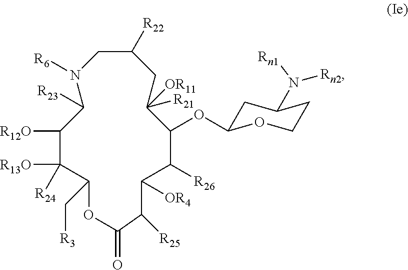
wherein, Rₙ₁, Rₙ₂, R₁₁, R₁₂, R₁₃, R₂₁, R₂₂, R₂₃, R₂₄, R₂₅, R₂₆, R₃, R₄, R₆, R₇ and R₈ are defined as above.

6. The compound of claim 1, wherein the compound of formula (I) is any of compounds listed in Table A.

7. A pharmaceutical composition comprising a compound of claim 1, or a pharmaceutically acceptable salt, a stereoisomer thereof; and a pharmaceutically acceptable carrier.

8. A use of a compound of claim 1, or a pharmaceutically acceptable salt, a stereoisomer thereof for manufacture of a medicament for treating or preventing inflammatory disease.

9. A method for treating or preventing inflammatory disease, which comprise a step of:
administering a compound according to claim 1 to a subject in need.

10. A method for promoting monocyte to macrophage in vitro, which comprises a step of: culturing cell in the present of a compound of claim 1.

11. A method for inhibiting the IL-₈ expression in vitro, which comprises a step of: culturing cell in the present of a compound of claim 1.
